(19) United States
(12) Patent Application Publication
(10) Pub. No.: US 2020/0276334 A1
(43) Pub. Date: Sep. 3, 2020

(54) New Therapy for Pompe Disease

(71) Applicant: Erasmus University Medical Center Rotterdam, Rotterdam (NL)

(72) Inventors: Wilhelmus Wenceslaus Matthias PIJNAPPEL, Rotterdam (NL); Antje Tjitske VAN DER PLOEG, Rotterdam (NL); Erik VAN DER WAL, Rotterdam (NL); Jur KRUIJT, Rotterdam (NL); Pablo HERRERO HERNANDEZ, Rotterdam (NL)

(21) Appl. No.: 16/645,561

(22) PCT Filed: Sep. 7, 2018
(86) PCT No.: PCT/NL2018/050581

§ 371 Date: Mar. 9, 2020

(30) Foreign Application Priority Data
Sep. 8, 2017 (NL) 2019517

(51) Int. Cl. A61K 48/00; C12N 15/85; C12N 5/074; A61K 35/12; A61K 9/00

(52) U.S. Cl. CPC A61K 48/005; C12N 15/85; A61K 9/0019; A61K 35/12; C12N 5/0607

(57) ABSTRACT

The invention relates to methods for gene therapy in a subject suffering from Pompe disease, comprising gene-editing of a glucosidase, acid, alpha gene (GAA) in said subject. The invention further relates to a cell culture of genetically changed, differentiated myogenic progenitor cells derived from a donor subject suffering from Pompe disease, to a vector for use in a method for gene-editing a eukaryotic cell, and to a myotube prepared from myogenic progenitor cells that have been genetically changed by gene-editing.

### FIELD OF THE INVENTION

The invention relates to a new therapy for Pompe disease.

### BACKGROUND OF THE INVENTION

Pompe disease, also known as acid maltase deficiency or glycogen storage disease type II, is an autosomal recessive metabolic disorder. It is caused by an accumulation of glycogen in the lysosome due to a deficiency of the lysosomal acid α-glucosidase enzyme. The build-up of glycogen occurs throughout the body and causes progressive skeletal muscle weakness (myopathy) and affects various body tissues, particularly in the heart, skeletal muscles, liver and nervous system.

In Pompe disease, a protein, α-D-glucoside glucohydrolase or, in short, acid α-glucosidase (EC 3.2.1.20, also known as acid maltase), which is a lysosomal hydrolase, is defective. The protein is an enzyme that normally degrades the α-1,4 and α-1,6 linkages in glycogen, maltose and isomaltose and is required for the degradation of 1-3% of cellular glycogen. The deficiency of this enzyme results in the accumulation of structurally normal glycogen in lysosomes and cytoplasm in affected individuals. Excessive glycogen storage within lysosomes may interrupt normal functioning of other organelles and lead to cellular injury.

A defective α-glucosidase protein, or reduced amount or activity of α-glucosidase protein, is the result of mutations (or variations) with in the Glucosidase, alpha, acid (GAA) gene. The GAA gene is located on the long arm of chromosome 17 at 17q25.2-q25.3 (base pairs 80,101,556 to 80,119,879 in GRCh38.p10). The gene contains 20 exons with the first exon being noncoding.

The Pompe Center at the Erasmus University in Rotterdam, the Netherlands, maintains an up-to-date catalog of the GAA mutations. Severe mutations that completely abrogate GAA enzyme activity cause a classic infantile disease course with hypertrophic cardiomyopathy, general skeletal muscle weakness, and respiratory failure and result in death within 1.5 years of life. Milder mutations leave partial GAA enzyme activity which results in a milder phenotype with onset varying from childhood to adult. In general, a higher residual enzyme activity in primary fibroblasts is associated with later onset of Pompe disease. Some of the GAA mutations in Pompe disease patients may lead to alternative splicing and thereby to absent or a reduced amount or activity of α-glucosidase protein. Although over 460 GAA mutations have been described (http://cluster15.erasmusme.n1/klgn/pompe/mutations.html), only a few splicing mutations have been characterized.

One of the most common mutations in Pompe disease is the IVS1 mutation, c.−32−13T>G, a transversion (T to G) mutation which occurs among infants, children, juveniles and adults with this disorder (Huie M L, et al., 1994. Hum Mol Genet. 3(12):2231-6). In childhood and adult Pompe disease, 90% of the patients in the Caucasian population are affected by the common c.32-13T>G (IVS1) variant that results in aberrant splicing of exon 2, such that exon 2 is partially or completely skipped. Absence of exon 2 from the mRNA results in absence of the normal AUG translation start site of the protein, which results in mRNA decay and failure to generate (AA protein. A small amount (10-15%) of splicing events escape skipping and occurs normally, leading to the production of normal GAA protein at ˜10-15% of levels found in healthy individuals.

Antisense oligonucleotides (antisense oligomeric compounds) are currently being tested in clinical trials for their ability to modulate splicing, for a number of disorders. With the IVS1 mutation, the development of antisense oligonucleotides is not straightforward. The IVS1 mutation causes an inclusion of a pseudoexon in intron 1, and a skipping of exon 2 resulting in the deletion of the canonical translation start side. The overall effects are enhanced RNA decay and absence of protein translation. For antisense therapy to work for the IVS1 mutation in Pompe disease, it needs to repress inclusion of a pseudo-exon in intron 1, and to induce inclusion of exon 2.

It has previously been shown that it is possible to promote exon inclusion in cells with the GAA IVS1 mutation using antisense oligonucleotides (WO2015/190921; WO 2015/190922; WO2017/099579). This presents a promising novel therapeutic option for Pompe patients. However, antisense oligonucleotides need to be taken life long and likely cannot restore existing tissue damage. To provide a life long therapy that is curative and can restore existing damage with a single intervention, a gene therapy approach may be required. However, correction of single mutations is highly inefficient, hampering clinical implementation of such approach. It is not obvious whether another, more efficient way to correct a single mutation such as the GAA IVS1 mutation would be possible.

Enzyme replacement therapy (ERT) has been developed for Pompe disease, in which recombinant human GAA protein is administered intravenously. This treatment is aimed to increase the intracellular level of α-glucosidase activity in affected cells and tissues and thereby reduce or prevent glycogen accumulation and eventually symptoms of the disease. The treatment can rescue the lives of classic infantile patients and delay disease progression of later onset patients, but the effects are heterogeneous. Also, it appears that there is a gradual decrease of the effect of added enzyme, and doses needs to be increased over time to maintain normal glycogen levels.

ERT has been used for treatment of infantile (infantile-onset or ‘classic infantile’), childhood (delayed-onset) and adult (late onset) Pompe patients. Targeting exogenous GAA protein to the main target tissues and cells is, however, a challenge. For example 15-40% of the body is composed of skeletal muscle and for the treatment to be effective each individual cell in the body needs to reached and loaded with exogenous GAA protein. The cells must take up the enzyme via endocytosis, which seems most efficient when receptors on the cell surface such as the mannose 6-phosphate/IGF II receptor are targeted. The mannose 6-phosphate/IGF II receptor recognizes various ligands such as mannose 6-phosphate, IGF II and Gluc-NAC. When these ligands are bound to (AA protein, a better uptake of the enzyme is obtained. The current registered ERT is targeted at the M6P part of the M6P/IGF II receptor, but there is also GAA protein ERT under development with an increased amount of M6P ligands or with IGF II linked to it.

In addition, ERT requires purified recombinant human GAA which is difficult to produce and therefore expensive. Furthermore, recombinant human GAA has a relative short half life ranging from 2-3 hours in blood to several days in cells and must therefore be administered intravenously every one to two weeks), which is cumbersome for patients. It is a further problem with ERT that accumulation of the GAA enzyme within target cells shows saturation, such that further increase of the external enzyme concentration does not result in higher intracellular enzyme concentrations. Furthermore, under clinical conditions using standard dosages, ERT results in levels and/or activities of intracellular GAA enzyme that are still lower than those observed in normal, healthy subjects, or obtained when using antisense therapy to induce exon inclusion in IVS1 mutations as shown. e.g., in Van der Wal et al. 2017 Molecular Therapy: Nucleic Acids Vol. 7:90-100.

An alternative for ERT would be to provide myogenic cells with a cDNA construct encoding normal human GAA protein, for example by incorporating an adenoviral associated vector encoding normal human GAA protein harbor into a “safe harbor” locus on the long arm of the human chromosome 19. However, targeting of such vector specifically to myogenic cells still is not straight forward. In addition, chromosomal insertion of the therapeutic DNA construct presently is unpredictable and requires selection using, for example, neomycin. Moreover, the consequence of overproduction of GAA protein in a cell in vivo is not known.

Another problem with the provision of a cDNA construct, and also with ERT, is that patients may develop antibodies to the exogenous GAA enzyme reducing the beneficial effects of and the treatment.

An alternative for in vivo application of ERT, the provision of a cDNA construct, and antisense oligonucleotides would be to genetically modify the GAA gene in myogenic cells of Pompe patients in vivo or ex vivo. Ex vivo modification is than followed by administration of the modified cells to a patient in need thereof. A major obstacle for this has been the lack of methods for reproducibly obtaining sufficient amounts of pure, preferably patient-derived, differentiated human skeletal muscle cells. It has not been possible to generate large amounts of isolated skeletal muscle cells from pluripotent stem cells, and to test therapies quantitatively. This is one of the major reasons that, as to date, very few therapies have been developed, as these therapies cannot be tested quantitatively in vitro.

Application of induced pluripotent stem (iPS) cells to model human disorders affecting skeletal muscle or as a basis for treatment of muscle disorders has predominantly been hampered by the difficulty to differentiate pluripotent stem cells into skeletal muscle cells in vitro and to obtain sufficient amounts of pure, patient-derived, differentiated human skeletal muscle cells. Alternatively, transgene overexpression of MyoD or Pax7, which are required for myogenic differentiation and which are markers for certain stages of myogenic differentiation, may be difficult. It is therefore an object of the invention to provide an improved therapy for treatment of Pompe disease patients, specifically forms of Pompe Disease in which a mutation affects correct splicing, and especially for treatment of Pompe disease with the GAA IVS1 mutation that causes skipping of exon 2.

### SUMMARY OF THE INVENTION

The invention therefore provides a method for gene therapy in a subject suffering from Pompe disease, comprising gene-editing of a glucosidase, acid, alpha gene (GAA) in said subject, which gene-editing comprises removal of a large part of intron 1 of the GAA gene, preferably wherein said gene-editing is specifically targeted at pluripotent stem cells or myogenic progenitor cells or myogenic cells.

A method of the invention provides an efficient way to correct endogenous expression of GAA without overexpression of exogenous GAA protein. A method of the invention may be combined with autologous stem cell transplantation, thereby obviating many of the negative aspects associated with ERT, the provision of a cDNA construct, and the use of antisense oligonucleotides.

In a preferred method of the invention, said subject is suffering from Pompe disease with IVS1 mutation c.−32−13T>G. It was surprisingly found that cells of a Pompe disease patient with IVS1 mutation c.−32−13T>G, showed enhanced expression of wild type GAA protein, despite the fact that the deletion not necessarily includes the IVS1 mutation itself. The observed enhanced expression was to such extent that it can not be explained only by enhanced correct splicing of the pre-mRNA, but seems also to involve enhanced transcription of the GAA gene, besides improved splicing.

The fact that transcription of the GAA gene seems to be enhanced by deletion of a large part of intron 1 of the GAA gene, makes is credible that such deletion can also be used in therapy of other forms of Pompe disease besides Pompe disease with IVS1 mutation c.-32-13T>G, especially of juvenile or adult forms of Pompe disease with residual expression of functional GAA protein. Elevation of expression in these patients, for example a two-fold elevation of the steady state of GAA protein in these patients, by removal of a large part of intron 1 of the GAA gene, may provide a strong, beneficial effect to these patients.

The invention further provides a method for gene therapy in a subject suffering from Pompe disease, comprising the steps of providing isolated myogenic progenitor cells, or pluripotent stem cells (PSC), that are obtained from the subject; removing a large part, of intron 1 of the GAA gene by gene editing to provide modified myogenic progenitor cells or PSC; optionally, in case of PSC, differentiate and expand these modified cells to obtain modified myogenic progenitor cells; and administering said modified myogenic progenitor cells to said subject.

The invention further provides a cell culture of genetically changed, differentiated myogenic progenitor cells, wherein said cells are derived from a donor subject suffering from Pompe disease, wherein said cells have been genetically changed by gene-editing, thereby removing a large part of intron 1 of the GAA gene, for use in the therapy of Pompe disease, wherein said cells are administered to the donor.

Further provided is a method according to the invention, or cell culture for use according to the invention, wherein the gene-editing has been achieved by integrating a viral vector, by action of a site-specific nuclease such as TALEN or ZFN, or by use of a CRISPR based nuclease, such as a Cas9 or Cpf1 enzyme, preferably by use of a Cas9 enzyme.

A preferred Pompe disease that is treated with a method according to the invention, or with a cell culture for use according to the invention, is characterized by incorrect splicing of exon 2, in particular a Pompe disease that is characterized by the IVS1 mutation c.−32−13T>G.

Said large part of the intron 1 of the GAA gene preferably comprises more than 50%, preferably more than 630%, more preferably more than 80%, more preferably more than 90% of said intron.

Differentiation and expansion of PSC in method of the invention preferably is performed according to the method described in co-pending PCT/NL2017/050298, published as WO 2017/196175. Said method preferably comprises the steps of culturing said PSC in a synthetic culture medium supporting differentiation of said PSC towards a myogenic cell lineage for (i) a first period of 3-8 days in the presence of between 2-5 microM, preferably about 3.5 microM, of CHIR99021, (ii) a second period of 5-20 days in the presence of 10-30 ng/ml of FGF2; and, optionally, ((iii) a third period of 10-20 days in the presence of insulin-transferrin-selenium-ethanolamine (ITS-X), to thereby provide a cell culture of pre-differentiated PSCs comprising myogenic progenitors cells; isolating from said cell culture at least one C-Met+ and Hnk1− myogenic progenitor starting cell, preferably by FACS, to thereby provide a purified myogenic cell lineage; expanding said at least one isolated C-Met+ and Hnk1− myogenic progenitor starting cell in a synthetic culture medium comprising fetal bovine serum (FBS), preferably in a concentration of about 10% (w/w), and 90-110 ng/ml of FGF2 for at least 1 passage, preferably at least 7 passages, to thereby provide a cell culture comprising a population of expanded C-Met+ and Hnk1− myogenic progenitor cells, wherein at least 50%, preferably at least 90% of said population of expanded C-Met+ and Hnk1− myogenic progenitor cells are myogenic marker MyoD positive and myogenic marker Pax7 negative, preferably wherein said expanding said at least one C-Met+ and Hnk1− myogenic progenitor starting cell is performed in a culture medium comprising a ROCK inhibitor during at least the culturing period prior to a first passage of the cells. The culture medium base preferably is DMEM-HG.

A method for gene therapy according to invention, and a cell culture for use according to the invention, preferably comprises the administration of the gene-edited myogenic progenitor cells by injection of said cells into a muscle of the subject. Said muscle preferably is injured prior to the administration of the gene-edited myogenic progenitor cells.

The invention further provides a vector for use in a method for gene-editing a eukaryotic cell, wherein said vector comprises a guide RNA sequence and wherein said gene-editing comprises removal of a large part of intron 1 of the GAA gene. Said vector preferably encodes a guide RNA targeted to intron 1 of the GAA gene, preferably wherein this vector comprises the sequences CCGTGGCCTGAGAGGGGCCCC and/or CCCTGCTGGAGCTTTTCTCGC.

The invention further provides a myotube prepared from myogenic progenitor cells, wherein said myogenic progenitor cells are characterized by a deletion within the GAA gene resulting from being genetically changed by gene-editing, thereby removing a large part, of intron 1 of the GAA gene, preferably wherein said deletion does not cover the basepair sequence responsible for the c.−32−13T>G (IVS1) mutation, more preferably wherein it starts at least 60 nucleotides upstream of the 3′ splice site of exon 2 and at least 10 nucleotides downstream of the 5′ splice site of exon 1.

### LEGENDS TO THE FIGURES

FIG. 1. Partial removal of 1921 bp in IVS1 and WT GAA minigenes increases expression and corrects splicing. (A) Outline of the effect of the IVS1 variant (c.−32−13T>G) on aberrant splicing of exon 2 of GAA. (B) Cartoon of removal of a large part of intron 1 of GA4 in genomic DNA. (C) Cartoon of the GAA minigene in the pcDNA3.1 vector containing a CMV promoter, exon 1, exon 2, exon 3 and a polyA sequence. The IVS1 variant was introduced in intron 1 and 1921 bp was removed. (D) Transfection of the minigene into HEK293T cells and RT-PCR on cDNA with primers spanning exon 1-exon 3. Red arrows indicate the normal transcript (N), splice variant 3 (SV3) and splice variant 2 (SV2). (E) RT-qPCR with specific primers against N, SV2 and SV3. mRNA expression was calculated using the deltaCT method. Data are means±standard deviation of three biological replicates. *p<0.05, **p<0.01 and ***p<0.001.

FIG. 2. Removal of 2.1 kb in intron 1 of GAA using CRISPR/Cas9 in patient-derived induced pluripotent stem cells. (A) Cartoon showing the reprogramming of human fibroblasts carrying the IVS1 variant (C.-32-13T>G) and the C.525delT into induced pluripotent stem cells (iPSCs), followed by gene editing using CRISPR/Cas9. (B) Two single guide RNA (sgRNA) sequences targeting intron 1 on the genomic sequence are indicated with a black bar. Indicated also is a PAM site. (C) Strategy for genotyping by amplifying wildtype (WT) and Δ intron 1 containing product (D) RT-PCR on the pool of iPSCs of patient 1 and patient 2, nucleofected with CRISPR/Cas9 with the two sgRNA sequences indicated in A. Arrows indicate the WT and A intron 1 products. (E) Genomic DNA analysis of individually picked iPSC colonies for the partial deletion of intron 1 using primers indicated in (C). (F) Number and % of positive clones from (E). (G) Sequence analysis of PCR products spanning the cleavage site from the selected iPSC clone from each patient from (E).

FIG. 3. Shortening of GAA intron 1 in iPSC derived skeletal muscle cells results in complete correction of aberrant GAA splicing, GAA mRNA expression and GAA enzyme activity. (A) Differentiation strategy to generate iPSC derived skeletal muscle cells. Purified myogenic progenitor cells were expanded and used as indicated in the experimental setup. (B) Differentiation of MPCs to myotubes of WT patient 1 and A intron 1 patient 1. Myotubes were Stained with α-MHC (red) and α-Myogenin (green) antibodies and nuclei (blue) were visualized with Hoechst. The fusion index is shown at the bottom and was calculated from five technical replicates and represented as means±standard deviation (C) Skeletal muscle cells were analyzed with RT-PCR using primers spanning exon 1-exon 3. Red arrows indicate N, SV3 and SV2 products. (D) Splice product-specific RT-qPCR on the same samples as in (B), calculated with the deltaCT method. (E) GAA enzyme activity on skeletal muscle cells differentiated for 4 days with or without removal of intron 1. The dotted line indicates the disease threshold GAA enzyme activity as determined previously (van der Wal et al., 2017). Data are means±standard deviation of three or two biological replicates. *p<0.05. **p<0.01 and ***p<0.001.

FIG. 4. Allele-specific PCR strategy to identify the targeted allele following gene editing with CRISPR/Cas9. (A) Cartoon of the PCR strategy to amplify the wild type product (primer 2 with primer 3) or the product containing the partial deletion of intron 1 (primer 1 with primer 3). (B) Pie chart showing the analysis of a subset of clones from FIG. 2E with product-specific PCR and sequencing. (C) Sequencing of the specific PCR products as indicated in (A) from the selected clone of patient 1 and patient 2 iPSCs.

FIG. 5. Outline of the generation of myogenic progenitors (MPCs) from human iPSCs and potential downstream applications. Reprogramming of human fibroblasts into iPSCs is achieved using OSKM (Oct4, Sox2, KLF4 and c-MYC). Myogenic differentiation is started after 5 days of plating iPSCs by switching to medium containing 3.5 μM CHIR99021 (GSK3b inhibition), to FGF2 containing medium on day 10, and in the last step to a basic medium until day 40. Differentiated cells are FACS-purified for C-MET⁺/Hoechst⁺/HNK⁻ cells and expanded (average cell division time is ˜32 hours). Myogenic progenitors can be used for in vitro differentiation into mature spontaneous contracting skeletal muscle cells, engrafted into immunodeficient mice or cryopreserved during the expansion phase.

FIG. 6. Purification of C-MET⁺/HNK1-⁻/Hoechst⁺ cells after 40 days of differentiation by FACS. Gates for C-MET⁺/Hoechst/HNK1⁻ population were defined with single stained or unstained cells. The horizontal line represents the average % MPCs of all samples in either Control or Pompe groups. Each symbol represents an individual differentiation experiment. The iPSC lines from different donors are indicated on the right.

FIG. 7. Myogenic potential of transplanted MPCs. (A) Four weeks after transplantation, engraftment of MPCs was determined by immunohistochemistry of human specific lamin A/C and spectrin and multi-species laminin. Top left panel: Tissue section with delimited area containing the engraftment. Top right panel: amplified area containing the engraftment. Middle panel: Same as top right panel with engrafted lamin A/C+ nuclei and spectrin+ myofibers in white. (B) Inserts represent two different locations of lamin A/C+ nuclei within the same spectrin+ muscle fiber. The human nuclei on a satellite cell position are indicated with an arrow and the myonuclei with an arrowhead. All sections were counterstained with Hoechst (blue). Scale bars represent 100 μm. (C) Numbers of spectrin+ muscle fibers and lamin A/C+ nuclei per section of each biological replicate. All sections were counterstained with Hoechst. Scale bars represent. 100 μm (n=2 biological replicates).

FIG. 8. Staining of human and mouse sections using human specific antibodies. Human muscle biopsis and mouse PBS injected sections were stained by immunohistochemistry with human specific lamin A/C and spectrin antibodies (white or red) and multi-species laminin. Hoechst was used to visualize the nuclei.

FIG. 9. Cell contribution to muscle regeneration in vivo. (A) Percentage of lamin A/C+ nuclei present at the muscle interstitium. (B) Percentage of spectrin+ fibers related to total number of lamin A/C+ nuclei. Numbers obtained per section of each biological replicate (n=2).

### DETAILED DESCRIPTION

The terms “individual”, “patient”, and “subject” are used interchangeably herein and refer to mammals, in particular primates and preferably humans. The term “exon” refers to a portion of a gene that is present in the mature form of mRNA. Exons include the ORF (open reading frame), i.e., the sequence which encodes protein, as well as the 5′ and 3′ UTRs (untranslated regions). The UTRs are important for translation of the protein. Algorithms and computer programs are available for predicting exons in DNA sequences (Grail, Grail 2 and Genscan and US 20040219522 for determining exon-intron junctions).

As used herein, the term “protein coding exon” refers to an exon which codes (or at least partially codes) for a protein (or part of a protein). The first protein coding exon in an mRNA is the exon which contains the start codon. The last protein encoding exon in an mRNA is the exon which contains the stop codon. The start and stop codons can be predicted using any number of well-known programs in the art.

As used herein, the term “internal exon” refers to an exon that is flanked on both its 5′ and 3′ end by another exon. For an mRNA comprising n exons, exon 2 to exon (n−1) are the internal exons. The first and last exons of an mRNA are referred to herein as “external exons”.

A “natural cryptic splice site” or “natural pseudo splice site” is a site that is normally not used in pre-mRNA splicing, but can be utilized when canonical splicing has been weakened. It can be located either in an intron or an exon. The term “induced splice site” refers to an RNA sequence that is changed by an (induced) mutation, resulting in the generation of a novel splice site that is used in pre-mRNA splicing.

The term “natural pseudo-exon” or “natural cryptic exon” refers to a region in the pre-mRNA that could function as an exon during splicing and is located in an intronic region of the pre-mRNA. The natural pseudo exon is not utilized in normal, healthy cells, but is utilized in diseased cells that carry a mutation in the gene. The strength of the natural cryptic splice sites is usually not affected by the presence or absence of such a mutation, although in some cases its predicted strength can change due to a nearby mutation.

The term “intron” refers to a portion of a gene that is not translated into protein and while present in genomic DNA and pre-mRNA, it is removed in the formation of mature mRNA.

The term ‘a large part of an intron’ refers to a part of at least 500 basepairs and at the largest 1 megabase (1 million basepairs). Preferably, the large part has a length of at least 1000 basepairs. Further, in the present invention a ‘large part of intron 1 of the GAA gene’ will preferably comprise the nucleotide portion that is responsible for the effect of the GAA c.−32−13T>G (IVS1) mutation on correct splicing of exon 2. More preferably said large part does not comprise the GAA c.−32−13T>G (IVS1) mutation itself. More preferably the deletion starts at least 60 nucleotides upstream of the 3′ splice site of exon 2 and at least 10 nucleotides downstream of the 5′ splice site of exon 1.

The term “messenger RNA” or “mRNA” refers to RNA that is transcribed from genomic DNA and that carries the coding sequence for protein synthesis. Pre-mRNA (precursor mRNA) is transcribed from genomic DNA. In eukaryotes, pre-mRNA is processed into mRNA, which includes removal of the introns, i.e., “splicing”, and modifications to the 5′ and 3′ end (e.g., polyadenylation). mRNA typically comprises from 5′ to 3′; a 5′cap (modified guanine nucleotide), 5′ UTR (untranslated region), the coding sequence (beginning with a start codon and ending with a stop codon), the 3′ UTR, and the poly(A) tail.

The terms “nucleic acid sequence” or “nucleic acid molecule” or “nucleotide sequence” or “polynucleotide” are used interchangeably and refer to a DNA or RNA molecule (or non-natural DNA or RNA variants) in single or double stranded form. An “isolated nucleic acid sequence” refers to a nucleic acid sequence which is no longer in the natural environment from which it was isolated, e.g. the nucleic acid sequence in a cell.

A “mutation” in a nucleic acid molecule is a change of one or more nucleotides compared to the wild type sequence, e.g. by replacement, deletion or insertion of one or more nucleotides. A “point mutation” is the replacement of a single nucleotide, or the insertion or deletion of a single nucleotide.

“Sequence identity” and “sequence similarity” can be determined by alignment of two peptide or two nucleotide sequences using global or local alignment algorithms. Sequences may then be referred to as “substantially identical” or “essentially similar” when they are optimally aligned by for example the programs GAP or BESTFIT or the Emboss program “Needle” (using default parameters, see below) and share at least a certain minimal percentage of sequence identity (as defined further below). These programs use the Needleman and Wunsch global alignment algorithm to align two sequences over their entire length, maximising the number of matches and minimising the number of gaps. Generally, the default parameters are used, with a gap creation penalty=10 and gap extension penalty=0.5 (both for nucleotide and protein alignments). For nucleotides the default scoring matrix used is DNAFULL and for proteins the default scoring matrix is Blosum62 (Henikoff & Henikoff, 1992, PNAS 89, 10915-10919). Sequence alignments and scores for percentage sequence identity may for example be determined using computer programs, such as EMBOSS (http://wwwv.ebi.ac.uk/Tools/psa/emboss_needle/). Alternatively sequence similarity or identity may be determined by searching against databases such as FASTA. BLAST, etc., but hits should be retrieved and aligned pairwise to compare sequence identity. A percentage sequence identity preferably is determined over the whole length of the peptide or nucleotide sequences. Two proteins or two protein domains, or two nucleic acid sequences are “highly homogenous” or have “substantial sequence identity” if the percentage sequence identity is at least 70%, 75%, 80%, 85%, 90%, 95%, 98%, 99% or more, preferably at least 90%, 95%, 98%, 99% or more (as determined by Emboss “needle” using default parameters, i.e. gap creation penalty=10, gap extension penalty=0.5, using scoring matrix DNAFULL for nucleic acids an Blosum62 for proteins). Such sequences are also referred to as ‘homologous sequences’ herein, e.g. other variants of a pre-mRNA or homologues or derivatives of antisense oligomeric compounds. It should be understood that sequences with substantial sequence identity do not necessarily have the same length and may differ in length. For example sequences that have the same nucleotide sequence but of which one has additional nucleotides on the 3′- and/or 5′-side are 100% identical when relating to the shared sequence part.

The term “hybridisation” as used herein is generally used to mean hybridisation of nucleic acids at appropriate conditions of stringency as would be readily evident to those skilled in the art depending upon the nature of the probe sequence and target sequences. Conditions of hybridisation and washing are well known in the art, and the adjustment of conditions depending upon the desired stringency by varying incubation time, temperature and/or ionic strength of the solution are readily accomplished. See, for example, Sambrook, J. et al., Molecular Cloning: A Laboratory Manual, 2nd edition, Cold Spring Harbor Press, Cold Spring Harbor. N.Y. 1989. The choice of conditions is dictated by the length of the sequences being hybridised, in particular, the length of the probe sequence, the relative G-C content of the nucleic acids and the amount of mismatches to be permitted, low stringency conditions are preferred when partial hybridisation between strands that have lesser degrees of complementarity is desired. When perfect or near perfect complementarity is desired, high stringency conditions are preferred. For typical high stringency conditions, the hybridisation solution contains 6×S.S.C., 0.01 M EDTA, 1×Denhardt's solution and 0.5% SOS, hybridisation Hybridisation is carried out at about 68° C. for about 3 to 4 hours for fragments of cloned DNA and for about 12 to about 16 hours for total eukaryotic DNA. For lower stringencies the temperature of hybridisation is reduced to about 42° C. below the melting temperature (TM) of the duplex. The TM is known to be a function of the G-C content and duplex length as well as the ionic strength of the solution.

The term “allele(s)” means any of one or more alternative forms of a gene at a particular locus, all of which alleles relate to one trait or characteristic at a specific locus. One allele is present on each chromosome of the pair of homologous chromosomes. These may be identical alleles of the gene (homozygous) or two different alleles (heterozygous).

The term “mutant allele” refers herein to an allele comprising one or more mutations in the sequence (mRNA. cDNA or genomic sequence) compared to the wild type allele. Such mutation(s) (e.g. insertion, inversion, deletion and/or replacement of one or more nucleotide(s)) may lead to the encoded protein having reduced in vitro and/or in vivo functionality (reduced function) or no in vitro and/or in vivo functionality (loss-of-function), e.g. due to the protein e.g. being truncated or having an amino acid sequence wherein one or more amino acids are deleted, inserted or replaced. Such changes may lead to the protein having a different conformation, being targeted to a different sub-cellular compartment, having a modified catalytic domain, having a modified binding activity to nucleic acids or proteins, etc, it may also lead to a different splicing event.

A “fragment” of the gene or nucleotide sequence or antisense oligomeric compound refers to any subset of the molecule, e.g., a shorter polynucleotide or oligonucleotide.

As used herein, the terms “precursor mRNA” or “pre-mRNA” refer to an immature single strand of messenger ribonucleic acid (mRNA) that contains one or more intervening sequence(s) (introns). Pre-mRNA is transcribed by an RNA polymerase from a DNA template in the cell nucleus and is comprised of alternating sequences of introns and coding regions (exons). Once a pre-mRNA has been completely processed by the splicing out of introns and joining of exons, it is referred to as “messenger RNA” or “mRNA,” which is an RNA that is completely devoid of intron sequences. Eukaryotic pre-mRNAs exist only transiently before being fully processed into mRNA. When a pre-mRNA has been properly processed to an mRNA sequence, it is exported out of the nucleus and eventually translated into a protein by ribosomes in the cytoplasm.

As used herein, the terms “splicing” and “(pre-)mRNA processing” refer to the modification of a pre-mRNA following transcription, in which introns are removed and exons are joined. Pre-mRNA splicing involves two sequential biochemical reactions. Both reactions involve the spliceosomal transesterification between RNA nucleotides. In a first reaction, the 2′-OH of a specific branch-point nucleotide within an intron, which is defined during spliceosome assembly, performs a nucleophilic attack on the first nucleotide of the intron at the 5′ splice site forming a lariat intermediate. In a second reaction, the 3′-OH of the released 5′ exon performs a nucleophilic attack at the last nucleotide of the intron at the 3′ splice site thus joining the exons and releasing the intron lariat. Pre-mRNA splicing is regulated by intronic silencer sequence (ISS), exonic silencer sequences (ESS) and terminal stem loop (TSL) sequences.

As used herein, the terms “intronic silencer sequences (ISS)” and “exonic silencer sequences (ESS)” refer to sequence elements within introns and exons, respectively, that control alternative splicing by the binding of trans-acting protein factors within a pre-mRNA thereby resulting in differential use of splice sites. Typically, intronic silencer sequences are less conserved than the splice sites at exon-intron junctions.

As used herein. “modulation of splicing” refers to altering the processing of a pre-mRNA transcript such that there is an increase or decrease of one or more splice products, or a change in the ratio of two or more splice products. Modulation of splicing can also refer to altering the processing of a pre-mRNA transcript such that a spliced mRNA molecule contains either a different combination of exons as a result of exon skipping or exon inclusion, a deletion in one or more exons, or additional sequence not normally found in the spliced mRNA (e.g., intron sequence).

As used herein, “splice site” refers to the junction between an exon and an intron in a pre-mRNA (unspliced RNA) molecule (also known as a “splice junction”). A “cryptic splice site” is a splice site that is not typically used but may be used when the usual splice site is blocked or unavailable or when a mutation causes a normally dormant site to become an active splice site. An “aberrant splice site” is a splice site that results from a mutation in the native DNA and pre-mRNA.

As used herein. “splice products” or “splicing products” are the mature mRNA molecules generated from the process of splicing a pre-mRNA. Alternatively spliced pre-mRNAs have at least two different splice products. For example, a first splicing product may contain an additional exon, or portion of an exon, relative to a second splicing product. Splice products of a selected pre-mRNA can be identified by a variety of different techniques well known to those of skill in the art (e.g. Leparc, G. G. and Mitra. R. D. Nucleic Acids Res. 35(21): e146, 2007).

As used herein “splice donor site” refers to a splice site found at the 5′ end of an intron, or alternatively, the 3′ end of an exon. Splice donor site is used interchangeably with “5′ splice site.” As used herein “splice acceptor site” refers to a splice site found at the 3′ end of an intron, or alternatively, the 5′ end of an exon. Splice acceptor site is used interchangeably with “3′ splice site.”

As used herein, “targeting” or “targeted to” refer to the process of designing an oligomeric compound such that the compound hybridizes with a selected nucleic acid molecule or region of a nucleic acid molecule. Targeting an oligomeric compound to a particular target nucleic acid molecule can be a multistep process. The process usually begins with the identification of a target nucleic acid whose expression is to be modulated. As used herein, the terms “target nucleic acid” and “nucleic acid encoding GAA” encompass DNA encoding GAA, RNA (including pre-mRNA and mRNA) transcribed from such DNA, and also cDNA derived from such RNA. For example, the target nucleic acid can be a cellular gene (or mRNA transcribed from the gene) whose expression is associated with a particular disorder or disease state, or a nucleic acid molecule from an infectious agent. As disclosed herein, the target nucleic acid encodes GAA. The GAA protein may be any mammalian enzyme, but it preferably is the human GAA.

The targeting process usually also includes determination of at least one target region, segment, or site within the target nucleic acid for the antisense interaction to occur such that the desired effect. e.g., modulation of expression, will result.

As used herein, “target mRNA” refers to the nucleic acid molecule to which the oligomeric compounds provided herein are designed to hybridize. In the context of the present disclosure, target mRNA is usually unspliced mRNA, or pre-mRNA. In the context of the present invention, the target mRNA is GAA mRNA or GAA pre-mRNA.

“Region” is defined as a portion of the target nucleic acid having at least one identifiable structure, function, or characteristic. Target regions may include, for example, a particular exon or intron, or may include only selected nucleotides within an exon or intron which are identified as appropriate target regions. Target regions may also be splicing repressor sites. Within regions of target nucleic acids are segments.

“Segments”, as used herein, are defined as smaller or sub-portions of regions within a target nucleic acid.

“Sites,” as used in the present invention, are defined as unique nucleobase positions within a target nucleic acid. As used herein, the “target site” of an oligomeric compound is the 5′-most nucleotide of the target nucleic acid to which the compound binds.

As used herein. “complementary” refers to a nucleic acid molecule that can form hydrogen bond(s) with another nucleic acid molecule by either traditional Watson-Crick base pairing or other non-traditional types of pairing (e.g., Hoogsteen or reversed Hoogsteen hydrogen bonding) between complementary nucleosides or nucleotides. In reference to the antisense oligomeric compound of the present disclosure, the binding free energy for an antisense oligomeric compound with its complementary sequence is sufficient to allow the relevant function of the antisense oligomeric compound to proceed and there is a sufficient degree of complementarity to avoid non-specific binding of the antisense oligomeric compound to non-target sequences under conditions in which specific binding is desired, i.e., under physiological conditions in the case of ex vivo or in vivo therapeutic treatment. Determination of binding free energies for nucleic acid molecules is well known in the art (see e.g., Turner et ah, CSH Symp. Quant. Biol. 1/7:123-133 (1987): Frier et al. Proc. Nat. Acad. Sci. USA 83:9373-77 (1986); and Turner et al, J. Am. Chem. Soc. 109:3783-3785 (1987)). Thus, “complementary” (or “specifically hybridizable”) are terms that indicate a sufficient degree of complementarity or precise pairing such that stable and specific binding occurs between a antisense oligomeric compound and a pre-mRNA or mRNA target. It is understood in the art, that a nucleic acid molecule need not be 100% complementary to a target nucleic acid sequence to be specifically hybridizable. That is, two or more nucleic acid molecules may be less than fully complementary. Complementarity is indicated by a percentage of contiguous residues in a nucleic acid molecule that can form hydrogen bonds with a second nucleic acid molecule. For example, if a first nucleic acid molecule has 10 nucleotides and a second nucleic acid molecule has 10 nucleotides, then base pairing of 5, 6, 7, 8, 9, or 10 nucleotides between the first and second nucleic acid molecules represents 50%, 60%, 70%, 80%, 90%, and 100% complementarity, respectively. Percent complementarity of an oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656). Percent homology, sequence identity or complementarity, can be determined by, for example, the Gap program (Wisconsin Sequence Analysis Package, Version 8 for Unix. Genetics Computer Group, University Research Park, Madison Wis.), using default settings, which uses the algorithm of Smith and Waterman (Adv. Appl. Math., 1981, 2, 482-489). “Perfectly” or “fully” complementary nucleic acid molecules means those in which all the contiguous residues of a first nucleic acid molecule will hydrogen bond with the same number of contiguous residues in a second nucleic acid molecule, wherein the nucleic acid molecules either both have the same number of nucleotides (i.e., have the same length) or the two molecules have different lengths.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence, resulting in exon-exon junctions at the site where exons are joined. Whereas Antisense OligoNucleotide (AON) therapy targeting exon-exon junctions can be useful in situations where aberrant levels of a normal splice product are implicated in disease, where aberrant levels of an aberrant splice product are implicated in disease, or where an overproduction of a particular splice product is implicated in disease. Aberrant fusion junctions due to rearrangements or deletions are also suitable targets for such therapy.

“Gene-editing” refers to the process of changing the genetic information present in the genome of a cell. This gene-editing may be performed by manipulating genomic DNA, resulting in a modification of the genetic information. Such gene-editing may or may not influence expression of the DNA that has been edited.

An “expression vector” is a replicon, such as plasmid, phage, virus, or cosmid, to which another DNA segment, i.e. an “insert”, may be attached so as to bring about the replication of the attached segment in a cell. A “vector” in the present application is an organism, such as viruses or bacteria that may be used to transfer nucleic acids, proteins and/or bacteria into another organism. Especially in the present invention a vector is used to transfer the crosslinked Cas9-DNA complex into the target cell.

A “target DNA” as used herein is a DNA polynucleotide that comprises a “target site” or “target sequence.” The terms “target site.” “target sequence,” “target protospacer DNA.” or “protospacer-like sequence” are used interchangeably herein to refer to a nucleic acid sequence present in a target DNA to which a DNA-targeting segment of a guide RNA will bind, provided sufficient conditions for binding exist. For example, the target site (or target sequence) 5′-GAGCATATC-3′ within a target DNA is targeted by (or is bound by, or hybridizes with, or is complementary to) the RNA sequence 5′-GAUAUGCUC-3′. Suitable DNA/RNA binding conditions include physiological conditions normally present in a cell. Other suitable DNA/RNA binding conditions (e.g., conditions in a cell-free system) are known in the art; see. e.g., Sambrook, supra. The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “non-complementary strand.” By “site-directed modifying polypeptide” or “RNA-binding site-directed polypeptide” or “RNA-binding site-directed modifying polypeptide” or “site-directed polypeptide” it is meant a polypeptide that binds RNA and is targeted to a specific DNA sequence. A site-directed modifying polypeptide as described herein is targeted to a specific DNA sequence by the RNA molecule to which it is bound. The RNA molecule comprises a sequence that binds, hybridizes to, or is complementary to a target sequence within the target DNA, thus targeting the bound polypeptide to a specific location within the target DNA (the target sequence).

By “cleavage” is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, a complex comprising a guide RNA and a site-directed modifying polypeptide is used for targeted double-stranded DNA cleavage.

“Nuclease” and “endonuclease” are used interchangeably herein to mean an enzyme which possesses endonucleolytic catalytic activity for DNA cleavage.

By “cleavage domain” or “active domain” or “nuclease domain” of a nuclease it is meant the polypeptide sequence or domain within the nuclease enzyme which possesses the catalytic activity for DNA cleavage. A cleavage domain can be contained in a single polypeptide chain or cleavage activity can result from the association of two (or more) polypeptides. A single nuclease domain may consist of more than one isolated stretch of amino acids within a given polypeptide.

The RNA molecule that binds to the site-directed modifying polypeptide and targets the polypeptide to a specific location within the target DNA is referred to herein as the “guide RNA” or “guide RNA polynucleotide” (also referred to herein as a “guide RNA” or “gRNA”). A guide RNA comprises two segments, a “DNA-targeting segment” and a “protein-binding segment.” By “segment” it is meant a segment/section/region of a molecule, e.g., a contiguous stretch of nucleotides in an RNA. A segment can also mean a region/section of a complex such that a segment may comprise regions of more than one molecule. For example, in some cases the protein-binding segment (described below) of a guide RNA is one RNA molecule and the protein-binding segment therefore comprises a region of that RNA molecule. In other cases, the protein-binding segment (described below) of a guide RNA comprises two separate molecules that are hybridized along a region of complementarity. As an illustrative, non-limiting example, a protein-binding segment of a guide RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and may include regions of RNA molecules that are of any total length and may or may not include regions with complementarity to other molecules.

The DNA-targeting segment (or “DNA-targeting sequence”) comprises a nucleotide sequence that is complementary to a specific sequence within a target DNA (the complementary strand of the target DNA) designated the “protospacer-like” sequence herein. The protein-binding segment (or “protein-binding sequence”) interacts with a site-directed modifying polypeptide. When the site-directed modifying polypeptide is a Cas9 or Cas9 related polypeptide (described in more detail below), site-specific cleavage of the target DNA occurs at locations determined by both (i) base-pairing complementarity between the guide RNA and the target DNA; and (ii) a short motif (referred to as the protospacer adjacent motif (PAM)) in the target DNA. For Cas9, a preferred PAM sequence that is located adjacent to the target sequence, is 5′-NGG.

The protein-binding segment of a guide RNA comprises, in part, two complementary stretches of nucleotides that hybridize to one another to form a double stranded RNA duplex (dsRNA duplex).

A guide RNA and a site-directed modifying polypeptide (i.e., site-directed polypeptide) form a complex (i.e., bind via non-covalent interactions). The guide RNA provides target specificity to the complex by comprising a nucleotide sequence that is complementary to a sequence of a target DNA. The site-directed modifying polypeptide of the complex provides the site-specific activity. In other words, the site-directed modifying polypeptide is guided to a target DNA sequence (e.g. a target sequence in a chromosomal nucleic acid; a target sequence in an extrachromosomal nucleic acid, e.g. an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment of the guide RNA.

In most embodiments, a guide RNA comprises two separate RNA molecules (RNA polynucleotides: an “activator-RNA” and a “targeter-RNA”, see below) and is referred to herein as a “double-molecule guide RNA” or a “two-molecule guide RNA.” In other embodiments, the guide RNA is a single RNA molecule (single RNA polynucleotide) and is referred to herein as a “single-molecule guide RNA.” a “single-guide RNA,” or an “sgRNA.” The term “guide RNA” or “gRNA” is inclusive, referring both to double-molecule guide RNAs and to single-molecule guide RNAs (i.e., sgRNAs).

A two-molecule guide RNA comprises two separate RNA molecules (a “targeter-RNA” and an “activator-RNA”). Each of the two RNA molecules of a two-molecule guide RNA comprises a stretch of nucleotides that are complementary to one another such that the complementary nucleotides of the two RNA molecules hybridize to form the double stranded RNA duplex of the protein-binding segment.

An exemplary two-molecule guide RNA comprises a crRNA-like (“CRISPR RNA” or “targeter-RNA”) molecule (which includes a CRISPR repeat or CRISPR repeat-like sequence) and a corresponding tracrRNA-like (“trans-activating CRISPR RNA” or “activator-RNA” or “tracrRNA”) molecule. A crRNA-like molecule (targeter-RNA) comprises both the DNA-targeting segment (single stranded) of the guide RNA and a stretch (“duplex-forming segment”) of nucleotides that forms one half of the dsRNA duplex of the protein-binding segment of the guide RNA. A corresponding tracrRNA-like molecule (activator-RNA) comprises a stretch of nucleotides (duplex-forming segment) that forms the other half of the dsRNA duplex of the protein-binding segment of the guide RNA. In other words, a stretch of nucleotides of a crRNA-like molecule are complementary to and hybridize with a stretch of nucleotides of a tracrRNA-like molecule to form the dsRNA duplex of the protein-binding domain of the guide RNA. As such, each crRNA-like molecule can be said to have a corresponding tracrRNA-like molecule. The crRNA-like molecule additionally provides the single stranded DNA-targeting segment. Thus, a crRNA-like and a tracrRNA-like molecule (as a corresponding pair) hybridize to form a guide RNA. A double-molecule guide RNA can comprise any corresponding crRNA and tracrRNA pair.

A single-molecule guide RNA comprises two stretches of nucleotides (a targeter-RNA and an activator-RNA) that are complementary to one another, are covalently linked (directly, or by intervening nucleotides), and hybridize to form the double stranded RNA duplex (dsRNA duplex) of the protein-binding segment, thus resulting in a stem-loop structure. The targeter-RNA and the activator-RNA can be covalently linked via the 3′ end of the targeter-RNA and the 5′ end of the activator-RNA. Alternatively, targeter-RNA and the activator-RNA can be covalently linked via the 5′ end of the targeter-RNA and the 3′ end of the activator-RNA.

The term “activator-RNA” is used herein to mean a tracrRNA-like molecule of a double-molecule guide RNA. The term “targeter-RNA” is used herein to mean a crRNA-like molecule of a double-molecule guide RNA. The term “duplex-forming segment” is used herein to mean the stretch of nucleotides of an activator-RNA or a targeter-RNA that contributes to the formation of the dsRNA duplex by hybridizing to a stretch of nucleotides of a corresponding activator-RNA or targeter-RNA molecule. In other words, an activator-RNA comprises a duplex-forming segment that is complementary to the duplex-forming segment of the corresponding targeter-RNA. As such, an activator-RNA comprises a duplex-forming segment while a targeter-RNA comprises both a duplex-forming segment and the DNA-targeting segment of the guide RNA. Therefore, a double-molecule guide RNA can be comprised of any corresponding activator-RNA and targeter-RNA pair.

The term “myogenic cell”, as used herein, refers to a cell that, during further differentiation, gives rise to or forms muscle tissue, preferably skeletal muscle tissue, such as myotubes. The term myogenic cell includes reference to and includes cells expressing one or more of the myogenesis markers Pax3, Pax7, MyoD and/or myogenin. Such expression is, for instance, determinable by immunostaining. The term includes reference to a myogenic progenitor cell, i.e. a cell that is not yet fully or terminally differentiated, and includes satellite cells (muscle stem cells), activated satellite cells and myoblasts. Preferably, myogenic cells are not pluripotent stem cells and do preferably not express pluripotency markers such as NANOG, OCT4, SSEA4, TRA-1-81 and/or TRA-1-60, at least not to high levels. Before the expansion step and during isolation, as described in the context of the invention, it is preferred that a myogenic cell is C-Met+ and Hnk1−. Preferably, the myogenic cell is a human cell. Markers that can be used to identify myogenic cells of the present invention in certain embodiments of aspects herein described include Pax7 and Pax3 before expansion and during isolation.

The terms “expansion” and “expanded”, as used herein, refer to the process, respectively, the result, of cell division, proliferation or multiplication that is accompanied by in an increase in cell number or cell count of a population of cells under cultivation. The term “expanded” in the context of the present invention, preferably includes reference to embodiments wherein cells are passaged.

The term “passaged”, as used herein, refers to the process of enzymatic dissociation of individual cells from colonies, by, for instance, trypsin, and the replacement of the culture medium by fresh medium, to thereby allow the further growth of mammalian cells in culture.

The term “cell culture”, as used herein, refers to an in vitro population of viable cells under cell cultivation conditions. i.e. under conditions wherein the cells are suspended in a culture medium that will allow their survival and preferably their growth, such as, for instance, a DMEM-based culture medium. The cell culture is usually comprised in a container holding the cell culture, referred to as a culture chamber, wherein sufficient exchange of gases such as oxygen and CO₂ is allowed between the cell culture and the atmosphere to support cell viability. The term “cell culture”, as used herein, includes reference to liquid forms, semi-solid forms, forms comprising extracellular matrix (ECM) protein as described below, and to frozen forms of the cell culture, and to single chamber as well as multi-chamber cultivation environments, such as, for instance, a plurality of wells in a multi-well plate. A cell culture of the invention, which comprises, is comprised in, or is present in a synthetic culture medium, is preferably provided in a container or plate for holding a cell culture. Said container or plate is preferably coated with extracellular matrix (ECM) protein. As an extracellular matrix (ECM) protein, one may use, for instance, extracellular matrix (ECM) gel, preferably ECM gel from Engelbreth-Holm-Swarm murine sarcoma fibrillar collagen (E6909 Sigma), and/or preferably a combination of (rat tail) 35 collagen type I and MaxGel ECM (E0282 Sigma), and/or fibrillar collagen, and/or a component of ECM gel, and/or a synthetic mimetic of an ECM component, and/or collagen type I, or a combination thereof. It is envisaged herein that the invention may also be defined in terms of a synthetic culture medium comprising a cell culture of the invention. In the same manner, the invention may also be defined as a container or plate comprising a synthetic culture medium that comprises a cell culture of the invention.

The term “culture medium”, as used herein, refers to a medium generally used in the culturing of mammalian, preferably human, cells, including pluripotent stem cells, such as iPS cells, and myogenic progenitor cells and differentiated cells in the context of the invention. Preferably, such a medium is based on Dulbecco's Modified Eagle Medium (DMEM) or Ham's F12 nutrient medium, or combinations of such basal media, optionally supplemented with compounds that prevent bacterial contamination, such as antibiotics, preferably in the form of a combination of penicillin/streptomycin/glutamine and/or compounds intended to reduce the amount of fetal bovine serum (FBS) in the medium, such as a combination of insulin, transferrin, selenium, and ethanolamine, commercially available under the name ITS-X from Thermo Fisher Scientific Inc., Waltham. Mass. USA. A “culture medium” is, in some embodiments, preferably serum-free. A “culture medium” in aspects of this invention may be a defined medium, but may also comprise animal serum components. In other preferred embodiments of aspects of this invention, the medium may comprise an animal serum. In other preferred embodiments of aspects of this invention, the medium is preferably animal serum-free. The use of media with serum, or serum-free media can depend on the phase of differentiation and the status of the myogenic cell in a method of this invention.

A “culture medium” in aspects of this invention may comprise feeder cells. Preferably mouse fibroblast feeders or immortalized human skin fibroblast feeder cells are used. In alternative preferred embodiments, a feeder layer-free medium may be preferred.

The term “pluripotent stem cell”, as used herein, refers to a cell with the capacity to differentiate to cell types characteristic of all three germ layers. Pluripotent stem cells can be characterized by expression of pluripotency markers, such as NANOG, OCT4. SSEA4, TRA-1-81 and TRA-1-60. It will be understood that the characterization of a pluripotent stem cell can be based on the characteristic expression of markers other than NANOG, OCT4, etc. The term pluripotent stem cell includes reference to iPS cells and embryonic stem (ES) cells. Preferably, the pluripotent stem cell is human, preferably the pluripotent stem cell is an iPS cell.

The term “induced pluripotent stem (iPS) cell”, as used herein, refers to a type of pluripotent stem cell that can be generated directly from an adult somatic cell such as a fibroblast cell, by inducing expression of specific genes. The term encompasses pluripotent cells, that, unlike embryonic stem cells, are derived from differentiated somatic cells, that is, cells other than a gamete, germ cell, gametocyte or undifferentiated stem cell, that has a narrower, more defined or limited differentiation potential and that in the absence of experimental manipulation could not give rise to all types of cells in the organism. The differentiated somatic cells can be induced or reprogrammed to become iPS cells. The original set of reprogramming factors (also dubbed Yamanaka factors) are the genes Oct4, Sox2, cMyc, and Klf4, iPS cells are morphologically similar to ES cells, having a round shape, large nucleolus and scant cytoplasm. Colonies of iPS cells are similar to colonies of ES cells. In addition, iPS cells preferably express one or more key pluripotency markers measurable by a person skilled in the art, including, but not limited to, NANOG, OCT4, SSEA4, TRA-1-81 and TRA-1-60.

The term “myotube”, as used herein, refers to a skeletal muscle fiber formed by the fusion of differentiated myogenic cells. Differentiation of myoblasts into myotubes is evidenced by increased fusion index, increased number of nuclei per myotube, and/or increased mRNA and protein expression of myogenic markers including myogenin and myosin heavy chain. Myosin heavy-chain expression may be determined by immunostaining using the MF20 monoclonal antibody by methods well known in the art. A fusion index (%) may be determined by dividing the number of nuclei within multinucleated myotubes by the total number of nuclei analyzed.

The term “myofiber”, as used herein, refers to a matured myotube that is contractible inter alia due to the presence of titin, sarcomeres, nicotinic acetylcholine receptors and/or calcium channels. Myofibers are preferably further characterized by the presence of a basal lamina. In some instances, when reference is made to myotubes that are matured in that they are contractible, reference to myofibers is intended.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a plant or an animal, such as a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The therapy will desirably be administered during the symptomatic stage of the disease, and in some cases after the symptomatic stage of the disease.

It was demonstrated by Raben et al. (Hum. Mol. Genet. 5(7), 995-1000, 1996) in an artificial minigene system that removal of a large part (90%) of intron 1 of the GAA gene increased expression of the wild-type transcript of acid alpha glucosidase. However, at that time it was not possible to edit the genomic information in living cells. Surprisingly, the current inventors now found that deletion of the majority of intron 1 is not only possible by gene editing living cells, either ex vivo or in vivo, but that the result shows an unexpected boost of the expression of the wild-type transcript to a full restoration of normal expression levels. This effect appears to be attributable to the sum of two different mechanisms: first of all, removal of the intron restores normal splicing and, secondly removal of the intron appears to enhance steady state levels of the wild-type mRNA.

The invention is thus related to a method for therapy of Pompe disease, especially a form of Pompe disease with residual activity of the glucosidase enzyme such as juvenile and adult Pompe disease. A preferred form of Pompe disease is a form in which the splicing is affected, especially splicing of exon 2. More particularly, a Pompe disease with mutations in intron 1 that cause skipping of exon 2, especially Pompe disease that is characterized by the IVS1 mutation. Such a therapy can be provided in different embodiments.

One of the embodiments is the in vivo gene therapy which includes performing a deletion in the genome of the target cell, where such a deletion is a deletion of a large part of intron 1 of the GAA gene. Methods for gene therapy, although perhaps not yet fully developed commercially, are known to the skilled person. Nowadays, such methods mainly are directed to in vivo adding genetic information to the cells of the subject by administration of viral vectors, preferable adenovirus associated virus (AAV)-based vectors carrying the genetic information which is used to complement the defective genetic information in the cells of the subject. However, such a therapy could also be used for removal of genetic information, since genetic information in eukaryotic cells may be changed through homologous recombination. In the present case homologous recombination may be used to remove a large part of intron 1 of the GAA gene, since this intron is not extremely large and thus homologous recombination by replacing the intron with a DNA stretch in which the intron is removed will be achievable. Alternatively, in stead of removal at least a large part of the intron, the intron, or a large part thereof, can be replaced by another stretch of DNA that would not lead to erroneous splicing of exon 2. Said other stretch of DNA preferably comprises a splicing enhancer that enhances inclusion of exon 2. When in the present application ‘removal of a large part of intron 1’ is used, it should be understood to mean either physical removal of a large part of intron 1 or replacement of a large part of intron 1 with a sequence (that may be shorter or even longer than the replaced sequence) that would not lead to erroneous splicing of exon 2.

In vivo gene therapy in which inhibition of the function of genomic DNA takes place may currently be readily achieved by using the CRISPR-Cas system (see below for a more detailed description of this system). In one embodiment this specific use of the CRISPR-Cas system is also indicated as CRISPRi and it may be used not only for inhibition of expression of genes, but it may also target to intronic regions and prevent processing of such an intronic region. In the present case this would then mean that the aberrant splicing which occurs on basis of the intronic sequences may be prevented.

In another embodiment the removal of a part of the intron may be achieved by non-homologous end joining or homology-directed repair at specific genetic locations. A number of different techniques to achieve such a recombination are known in the art.

In a particular embodiment removal of part of the intron is achieved by integrating viral approaches such as (partial) gene replacement with viral vectors, such as adenoviral vectors. In an alternative embodiment, removal of a part of the intron is achieved by gene editing techniques on basis of the site specific creation of double stranded breaks (DSBs) caused by application of zinc finger nucleases (ZFN), transcription activator-like effector (TALE) nucleases or the gene editing that is based on the CRISPR sequences (Gaj, T. et al., Trends Biotechnol. 31(7):397-405, 2013).

The modular structure of zinc-finger proteins has made them an attractive framework for the design of custom DNA-binding proteins. Key to the application of zinc-finger proteins such as ZFN for specific DNA recognition was the development of unnatural arrays that contain more than three zinc-finger domains. This advance was facilitated by the structure-based discovery of a highly conserved linker sequence that enabled construction of synthetic zinc-finger proteins that recognized DNA sequences 9 to 18 bp in length. Because 18 bp of DNA sequence can confer specificity within 68 billion bp of DNA, this method allowed for specific sequences to be targeted in the human genome.

Transcription activator-like effector (TALE) proteins are naturally occurring proteins from the plant pathogenic bacteria genus Xanthomonas, and contain DNA-binding domains composed of a series of 33-35 amino acid repeat domains that each recognizes a single base pair. TALE specificity is determined by two hypervariable amino acids that are known as the repeat-variable di-residues (RVDs). Like zinc-fingers, modular TALE repeats are linked together to recognize contiguous DNA sequences. However in contrast to zinc finger proteins, re-engineering of the linkage between repeats is not necessary to construct long arrays of TALEs with the ability to theoretically address single sites in the genome.

In the past decades numerous effector domains have been made available to fuse to zinc fingers or to TALE repeats for targeted genetic modifications, including nucleases, transcriptional activators and site-specific recombinases. While the single base recognition of TALE-DNA binding repeats affords greater design flexibility than triplet-confined zinc-finger proteins, the cloning of repeat TALE arrays has presented a challenge due to extensive identical repeat sequences. To overcome this issue, several methods have been developed that enable rapid assembly of custom TALE arrays. These strategies include “Golden Gate” molecular cloning (Cermak. T. et al., Nucl. Acids Res. 39:e82, 2011), high-throughput solid-phase assembly (Reyon, D. et al., Nat. Biotechnol. 30:460-465, 2012) and ligation-independent cloning techniques (Schmid-Burg, J. L. et al., Nat. Biotechnol. 31:76-81, 2013). Several large-scale, systematic studies utilizing various assembly methods have indicated that TALE repeats can be combined to recognize virtually any user-defined sequence.

The use of site-specific nucleases for therapeutic purposes represents a paradigm shift in gene therapy. Unlike conventional methods, which either temporarily address disease symptoms or randomly integrate therapeutic factors in the genome. ZFNs and TALENs are capable of correcting the underlying cause of the disease, therefore permanently eliminating the symptoms with precise genome modifications.

However, the nowadays most successful and most widespread way of gene editing is based on the CRISPR (Clustered Regulatory Interspaced Short Palindromic Repeats)/CRISPR-associated (Cas) system. In the Type II CRISPR/Cas system, short segments of foreign DNA, termed “spacers” are integrated within the CRISPR genomic loci and transcribed and processed into short CRISPR RNA (crRNA s). These crRNAs anneal to trans-activating crRNAs (tracrRNAs) and direct sequence-specific cleavage and silencing of pathogenic DNA by Cas proteins. Cas9 (CRISPR associated protein 9) is an RNA-guided DNA endonuclease enzyme. Cas9 has gained traction in recent years because it can cleave nearly any sequence complementary to the crRNA. The target specificity of Cas9 stems from the crRNA:DNA complementarity and not on modifications to the protein itself (like TALENs and Zinc-fingers). Hence, engineering Cas9 to target non-self DNA is straightforward. While native Cas9 requires a so-called guide RNA composed of the two disparate RNAs that associate to make the guide—the CRISPR RNA (crRNA), and the trans-activating RNA (tracrRNA)—Cas9 targeting has been simplified through the engineering of a chimeric single guide RNA. Scientists have suggested that Cas9-based gene drives may be capable of editing the genomes of entire populations of organisms. In 2015, scientists used Cas9 to modify the genome of human embryos for the first time and in the most recent years the system has become immensely popular and widespread for gene editing of all kinds of eukaryotic cells. Next to Cas9 also other enzymes, such as Cpf1 and other Cas enzymes would be applicable in the present invention as long as they would cause site directed DSBs.

In a preferred embodiment of the present invention the removal of a large part of intron 1 of the GAA gene is achieved by using a CRISPR-Cas9 construct.

A large part of intron 1 of the GAA gene is defined in the present invention as more than 50% preferably more than 60%, more preferably more than 70%, more preferably more than 80% and most preferably about 90% or more of the intron. It is desirable when removing the intron to at least include a region comprising at least nucleotides c−32−209 to c−32−165. Said region comprising at least nucleotides c−32−209 to c−32−165 preferably extends towards exon 1 and has a length of about 0.5 kb, about 1 kb, about 1.5 kb, preferably about 2 kb such as about 2.1 kb. Said deletion preferably is from nucleotide c.−32−1240 to nucleotide c.−32−738, resulting in a deletion of about 500 bp, from nucleotide c.−33+1093 to nucleotide c.−32−571, resulting in a deletion of about 1000 bp, from nucleotide c.-33+760 to nucleotide c.−32−404, resulting in a deletion of about 1500 bp, or from nucleotide c.−33+427 to nucleotide c.−32−237, resulting in a deletion of about 2000 bp. Said region preferably is a region in between .−33+336-c.−32−191 of intron 1 of GAA, or the entire intron 1 of GAA, in order to prevent leaving behind sites that would cause erroneous splicing of exon 2 of the GAA gene. It is noted that the part, of intron 1 of the GAA gene that is to be removed does not necessarily include the IVS1 mutation c.−32−13T>G.

In a particular embodiment muscle cells or muscle progenitor cells are provided to a subject in need thereof, where the genetic information in said cells has been changed in such a way that a large part of intron 1 of the GAA gene is removed. Said cells can be provided in vivo or ex vivo. When applied in vivo the constructs that would serve for the intron removal would preferably be targeted at cells of the myogenic lineage, preferably myogenic progenitor cells and/or cells that can develop into myogenic progenitor cells, such as induced pluripotent stem cells (iPS). Targeting of constructs towards myogenic cells may be achieved by connecting the vector that is used to carry the sequence information for the intron removal to a targeting moiety that recognizes a marker on such cells, such as NANOG, OCT4, SSEA4. TRA-1-81, TRA-1-60, C-Met, MyoD, PAX-3 and/or PAX-7.

When applied ex vivo, iPS cells and/or myogenic progenitor cells are provided in a cell culture and the vector carrying the genetic information for the removal of a large part of the intron may be added to the culture medium, where after the cells are transfected or nucleofected according to standard techniques, well known to the skilled person, which e.g. are described in the experimental part of the present description.

Preferably, iPS cells are obtained by a method chosen from a multitude of viral and non-viral methods, including methods using adenovirus, plasmids, or excision of reprogramming factors using Cre/LoxP or piggyBAC transposition, methods using episomal vectors derived from the Epstein-Barr virus, or methods using a minicircle DNA vector (US Patent Publn. 2015/0183141). The vector comprises one or a plurality of sequences encoding reprogramming factors. In some embodiments the vector comprises a plurality of reprogramming factor-coding sequences, where the combination of factors present on the single vector is sufficient to induce pluripotency. The plurality of coding sequences may be operably linked to a single promoter, where coding sequences are separated by self-cleaving peptide sequences. A non-limiting example of factors sufficient to reprogram a somatic cell to pluripotency is: Oct4, Sox2, Lin28, and Nanog. An alternative non-limiting example of factors sufficient to reprogram a somatic cell to pluripotency is: Oct4, Sox2, c-Myc, and Klf4. In some embodiments, the vectors are optimized to remove expression-silencing bacterial sequences, where in many embodiments the vectors include a unidirectional site-specific recombination product sequence in addition to an expression cassette. In other methods of the invention, a population of human somatic cells is contacted with a cocktail of reprogramming factors, and maintained in a culture medium for a period of time sufficient to reprogram said human somatic cells to pluripotency. Methods of producing PSCs, such as e.g. iPSCs, are described in detail in for instance U.S. Pat. Nos. 8,058,065; 8,129,187; 8,211,697; 8,257,941; 8,278,104; 8,546,140; 8,791,248; 9,175,268; U.S. Patent Publn. Nos. 2008/0003560; 2008/0233610; 2010/0184227; 2013/0029866; 2013/0040302; 2013/0130387; EP 2 072 618 A1; WO/2009/032194; WO/2009/032456; WO/2010/042490; and WO/2010/077955, the contents of which are incorporated herein by reference in their entirety.

Any and all methods for generating PSCs are considered to be suitable for use in aspects of this invention and their inclusion herein is expressly foreseen. Such methods include lentiviral methods, retroviral methods, methods using Sendai virus, methods using protein transduction/nucleofection, as well as the method referred to as the iTOP method (D'Astolfo et al., 2015. Cell. Vol 161(3):674-90).

For reprogramming of somatic cells, in order to produce PSCs, such as e.g. iPSCs, certain aspects of the present methods may involve using the reprogramming factors sufficient to convert the somatic cell to a pluripotent stem cell when such factors are expressed in the somatic cell under appropriate cell culture conditions. For example, the reprogramming factor(s) can comprise one or more selected from the group consisting of Sox, Oct, Nanog, Lin-28, Klf4, C-myc, L-myc and SV40LT, for example, a set of Sox, Oct, Nanog, and optionally Lin-28, a set of Sox, Oct, Klf4, and optionally C-myc, or a combination of these factors. In certain aspects, to reduce the potential toxic effect of C-myc expression, the SV40 large T gene (SV40LT) may be included with c-Myc. In certain aspects to further improve reprogramming efficiency, Myc mutants, variants or homologs that are deficient in transformation may be used. Non-limiting examples include a Myc proto-oncogene family member such as LMYC (NM-001033081), MYC with 41 amino acid deleted at the N-terminus (dN2MYC), or MYC with mutation at amino acid 136 (W 136E).

In certain aspects, the somatic cells for use in embodiments according to the invention are primary human cells, which are cells directly obtained from a living human subject, and may exclude the use of an established and/or immortalized cell line. Some aspects can comprise the use of terminally differentiated human cells. Non-limiting examples of the primary human cell include a fibroblast, a keratinocyte, a hematopoietic cell, a mesenchymal cell, an adipose cell, an endothelial cell, a neural cell, a muscle cell, an epithelial cell, a mammary cell, a liver cell, a kidney cell, a skin cell, a digestive tract cell, a cumulus cell, a gland cell, a pancreatic islet cell or cells present in urine, saliva (e.g. of the salivary gland), sputum or in snot. More specifically, the primary human cell may be a hematopoietic progenitor cell, such as a CD34+ cell. The primary human cell may be obtained from a blood sample, a hair sample, a skin sample, a saliva sample, a solid tissue sample or any sources known to a person of ordinary skill in the art. In preferred aspects of the present invention, use may suitably be made of dermal fibroblasts obtained via skin biopsy as the somatic cells that are reprogrammed. In other embodiments of aspects of this invention, use can be made of any and all cell types amenable to reprogramming into a pluripotent stem cell, including, but not limited to cells from diseased patients, as well as cells from healthy subjects, including fibroblasts, keratinocytes, cells from blood, cells from urine, cells from salivary fluid, cells from a muscle biopsy, pericytes, mesoangioblasts, lymphocytes, teeth cells, hair cells, etc.

In certain aspects, culturing cells under reprogramming conditions comprises culturing the cells in a reprogramming medium. For example, a reprogramming medium may comprise one or more signaling inhibitor(s) (e.g., an inhibitor that has been added to the medium). The signaling inhibitors may be one or more selected from the group consisting of a glycogen synthase kinase 38 (GSK-3B) inhibitor, a mitogen-activated protein kinase kinase (MEK) inhibitor, a transforming growth factor beta (TGF-ß) receptor inhibitor, leukemia inhibitory factor (LIF), and a combination thereof. Particularly, the reprogramming medium can comprise a combination of GSK-3ß inhibitor. MEK inhibitor, TGF-ß receptor inhibitor, and optionally, LIF. In aspects of this invention, suitable cultivation conditions for reprogramming further include cultivating cells in a medium wherein cells are subjected to the 2i/LIF condition of dual inhibition (2i) of mitogen-activated protein kinase signaling and glycogen synthase kinase-3 (GSK3) with leukaemia inhibitory factor (LIF). The medium may further comprise externally added ROCK inhibitor or Myosin II inhibitor. The ROCK inhibitor may be HA-100 and/or Y-27632 dihydrochloride. The medium in aspects of the present invention further comprises as a supplement fibroblast growth factor 2 (FGF2). In certain aspects, the medium of the present invention may be a chemically defined medium. Non-limiting examples of a chemically defined media include mTeSR™1 or TeSR™-E7™ (STEMCELL Technologies SARL, Grenoble, France), Dulbecco's Modified Eagle's Medium (DMEM: ATCC® 30-2002), NBF medium (DMEM/F12 supplemented with N2, B27, and basic fibroblast growth factor: Liu et al., 2006, Biochem. Biophys. Res. Comm. 346(1):131-139), Essential 8™ medium (Thermo Fisher Scientific Inc.), Primate ES Cell Medium (ReproCELL, Inc., Yokohama, Japan), and derivatives thereof. Further methods for reprogramming of somatic cells are detailed in U.S. Patent Publn. 2011/0104125, incorporated herein by reference in its entirety.

In still further aspects, methods according to the embodiments comprise culturing cells in the presence of feeder cells, such as irradiated, or mitomycin C-treated, mouse embryonic fibroblast (MEF) feeder cells. Alternatively, cells may be cultured in conditions essentially free of feeder cells. For example, a method according to the invention may comprise culturing cells in the presence of a matrix component to replace feeder cells to support culture of the cell population. Such a matrix component for cell adhesion can be any material intended to attach stem cells or feeder cells (if used). Non-limiting examples of the matrix component include collagen, gelatin, poly-L-lysine, poly-D-lysine, vitronectin, laminin, and fibronectin and mixtures thereof, for example, Matrigel™ and lysed cell membrane preparations. In a particular example, the matrix composition includes a fibronectin fragment, such as RetroNectin® (see, e.g., U.S. Pat. Nos. 5,686,278; 6,033,907; 7,083,979 and 6,670,177, incorporated herein by reference in their entirety).

In some aspects, culturing of cells under reprogramming conditions comprises culturing the cells for at least from about one day, one week or one month under reprogramming conditions. For example, the cells can be cultured in a reprogramming medium (e.g., a medium comprising signaling inhibitors as described above) for at least or about 1, 2, 3, 4, 6, 7, 8, 9, 10, 11, 12, 13, 14, 15, 16, 17, 18, 19, 20, 25, 30, or 35 days or more, or any range derivable therein.

In yet further aspects of the embodiments culturing the cells under reprogramming conditions may further optionally comprise selecting or screening the cells for the presence of pluripotency markers or differentiation markers. For example, the cells can be selected or screened by fluorescence activated cell sorting (FACS), magnetic activated cell sorting (MACS) or flow cytometry. Alternatively or additionally, the cells may comprise a drug resistance marker and the cells can be selected by addition of an appropriate drug to the cell culture medium (e.g., puromycin). Accordingly, in certain aspects, culturing the cells under reprogramming conditions comprises culturing the cells in a reprogramming medium. For example, cells can be cultured about 1 to 10 days (e.g., about 1 to 2 days, 1 to 3 days or 1 to 5 days). Likewise, in certain aspects, the cells are cultured for at least about 1 to 10 days, such as for about 5, 10, 15, 20, 25, 30 or more days. Cells may be cultured at least until PSCs are produced.

In yet a further aspect, the methods of the embodiments may further comprise selecting PSCs, for example, based on one or more embryonic cell characteristics, such as an ES cell-like morphology. Thus, in still further embodiments, a method comprises selecting pluripotent cells based on the expression of at least a first marker of pluripotency. For example, a population of cells that express at least a first marker of pluripotency (e.g., Tra-1-60) can be isolated by picking of a clonal cell colony or by FACS. The pluripotent population can optionally be further separated as required.

The vector that is used for transfection or nucleofection can be any vector suitable for introducing nucleotide sequences into eukaryotic cells. Preferably, the vector is a vector that comprises or encodes at least a single guide RNA. Said guide RNA contains sequences that target intron 1 of the GAA gene. Preferably said target sequences are located near protospacer adjacent motifs (PAM) sequences for recognition by Cas9. Preferred target sequences are TGGCCTGAGAGGGGGCCCC and/or TGCTGGAGCTTTTCTCGC, more preferred CCGTGGCCTGAGAGGGGGCCCC and/or CCTGCTGGAGCTTTTCTCGC, which more preferred sequences comprise Cas9 PAM sequences.

The at least one guide RNA sequence is reverse complementary to the at least one target sequence and may be complexed with an endonuclease, preferably Cas9, through the presence of a scaffold sequence on the same RNA molecule. Said vector, or an additional vector, preferably encodes Cas9. As an alternative, an endonuclease, preferably Cas9, may be provided as mRNA or as protein to a myogenic cell by viral transduction, nucleofection, or any other method known in the art.

A preferred vector for in vivo or ex vivo deletion of a large part of intron 1 of GAA, preferably employing CRISPR/Cas, is a adenovirus-based vector or adenovirus associated virus-based vector, for example as described in Maggio et al., 2014 (Maggio et al., 2014. Scientific Reports 4: 5105), or as available from System Biosciences (Palo Alto, Calif., USA).

In a further embodiment of the invention provided are cells or cell cultures harbouring genetically modified cells that are treated ex vivo with the above described techniques. Such a cell culture comprising cells can be obtained by expanding the culture of transfected or nucleofected PSCs, obtained as described above. Examples of suitable expansion media include, but are not limited to, any of the media for cell reprogramming described above, normal ES cell culture medium (DMEM supplemented with 15% FBS, 0.1 mM non-essential amino acids, 0.1 mM 2-mercaptoethanol, 2 mM Glutamine, 100 U/ml penicillin/streptomycin and 1000 U/ml LIF). Such a method for culturing and expanding PSCs and especially iPSCs has been extensively described in co-pending application WO 2017/196175.

Said cells or cell cultures harboring genetically modified cells comprising a deletion of a large part of the first intron of GAA are preferably obtained from a donor subject suffering from Pompe disease, preferably Pompe disease with a mutation that causes skipping of exon 2 such as, for example, IVS1, and are preferably administered to the same donor subject after expansion of the cells and, when necessary, differentiation of the cells into myogenic cells.

Differentiation of the pluripotent stem cells towards a myogenic lineage may be effected by culturing the pluripotent stem cell with (i) a Wnt agonist and/or a glycogen synthase kinase 3 beta (GSK3B) inhibitor, and (ii) an FGF pathway activator. Preferably, PSC's are cultivated in the presence of a relatively high concentration of a GSK3B inhibitor such as CHIR99021 (6-[2-[[4-(2,4-dichlorophenyl)-5-(5-methyl-1H-imidazol-2-yl)pyrimidin-2-yl]amino]ethylamino]pyridine-3-carbonitrile). The amount of the GSK3B inhibitor in the pre-differentiation medium is preferably higher than 3 μM, more preferably at least 3.2 μM or at least 3.5, 4, 5, 6, 7, 8, 9, or 10, or 15 μM. Surprisingly, such high concentrations of GSK3B inhibitors are well tolerated by PSCs. Preferably, the duration of culturing in the presence of a GSK3B inhibitor is 5-6 days, or a longer duration (e.g. 5-10 days,). Alternatively, a production of a myogenic cell lineage can be obtained by culturing cells with a GSK3B inhibitor at a concentration of 2-15 μM, preferably for a period of 2-10 days.

The in vitro differentiation of PSC cells to myogenic cells further comprises a second period of culturing the cells with a FGF pathway activator, preferably FGF2, at a concentration of 5-50 ng/ml, preferably 10-30 ng/ml, more preferably about 20 ng/ml, of FGF2. Preferably, the GSk3B inhibitor is not present anymore during the second period of culturing. The second period of culturing can be variable, but is preferably between 5-20 days, preferably 10-16 days.

Preferably, the in vitro differentiation of PSC cells to myogenic cells further involves a third period of culturing with a differentiation medium that pushes the cells further into the mesoderm orientation, i.e. towards a myogenic cell lineage positive for myogenesis marker Pax3 and/or Pax7. A suitable example of such a medium is DMEM/F12, ITS-X, such as 1×ITS-X, and Penicillin/Streptomycin-Glutamine (preferably all Gibco™).

The third period of culturing is preferably 5-30 days, more preferably 10-20 days, and most preferably 14-18 days. C-Met+ and Hnk1− expressing cells should be isolated from said cell culture to obtain an enriched cell culture comprising myogenic progenitors cells to thereby provide a myogenic cell lineage.

Alternatively, or in addition, it is envisaged that the step of isolating cells, or isolated cells as such may refer to the isolation of, or to an isolated, Hnk1− and AchR+ (acetylcholine receptor) myogenic progenitor cell(s). Selection and isolation of the cell lineage of interest preferably should occur prior to expansion. Expansion after selection is very beneficial in that expanded cultures of pure, isolated, transfected or nucleofected cells can be obtained, which provides for very homogeneous lineages of myogenic cells.

Expansion can then be achieved by culturing the myogenic progenitor cells in a culture medium comprising an FGF pathway activator to thereby provide a cell culture of expanded myogenic progenitor cells. The concentration of the FGF pathway activator in the expansion medium is suitably 25-200 ng/ml, preferably 70-150 ng/ml, more preferably 90-110 ng/ml, most preferably about 100 ng/ml. Preferably, the FGF pathway activator is FGF2. To this medium optionally 100 U/ml Penicillin/Streptomycin/Glutamine and/or 10% fetal bovine serum is added. Expansion of isolated myogenic progenitor cells, in the first stage of expansion, i.e. prior to the first passage, and preferably also during subsequent passages, is preferably performed using a medium comprising a Rho Kinase inhibitor (ROCK inhibitor). Suitable Rho Kinase inhibitors include RevitaCell™ Supplement (Gibco™). HA-100, Y-27632 dihydrochloride and Thiazovivin or a combination thereof. RevitaCell™ Supplement is preferred.

The process of expansion can be performed on a single, isolated, transfected or nucleofected myogenic cell or on a plurality of isolated, transfected or nucleofected myogenic cells. Preferably, the expanded myogenic progenitor cells are clonal cells, meaning that they are derived from a single transfected or nucleofected PSC cell. This ensures that such cells are genetically identical.

The expanded genetically altered cell culture of myogenic cells according to the present invention can be in frozen form. The expanded cells are preferably taken up in a cryopreservation medium when they are frozen. Suitable cryopreservation media or methods of cryopreserving human stem cells are described in the art, such as for instance in US 20050026133 A1 or WO 2005118785 A1.

It is further possible to differentiate the expanded, transfected or nucleofected myogenic progenitor cells to form differentiated human skeletal muscle cells and/or myotubes, and optionally allowing said myotubes to mature to muscle fibers. In order to produce myotubes from myogenic cells one may use any one of the classical techniques for induction of terminal differentiation, e.g. by deprivation of serum in the medium, such as described by Yoshida et al. (J Cell Science 111, 769-779 (1998)), through formation of PSM-like cells as described in Chal et al. (Nature Biotechnology 33, 962-969 (2015)), or using the two-dimensional muscle syncytia (2DMS) technique of Yamamoto et al. (J Histochem Cytochem, 56(10): 881-892 (2008)), all incorporated herein by reference).

Cells and fused cell structures, such as myotubes or myofibers, produced according to the methods of this invention, can be used for engrafting muscles of subjects suffering from Pompe disease. As is shown in the experimental part, transplants of these myotubes are well incorporated in mammalian muscle tissue.

Preferred routes of administration of genetically altered, differentiated myogenic progenitor cells to a donor subject include intravenous, intra-arterial, intramuscular, subcutaneous, intraperitoneal, spinal or other parenteral routes of administration, executed inter alia by injection or infusion. Administration can be performed, for example, once, a plurality of times, and/or over one or more extended periods of time. It is anticipated that myogenic cells, after administration of a genetically altered cell culture of myogenic cells to a subject in need thereof, will fuse with existing skeletal muscle fiber or form new skeletal muscle fibers to participate in generating skeletal muscle tissue.

A preferred parenteral route is intramuscular injection.

Prior to administration of genetically altered, differentiated myogenic progenitor cells to a donor subject, a muscle of the donor subject may be treated to pre-condition the host muscle for transplantation. Said pre-condition step is aimed at inducing a muscle injury, thereby elucidating regeneration of muscle in which the genetically altered, differentiated myogenic progenitor cells will participate.

Before administration of genetically altered, differentiated myogenic progenitor cells, preferably about 1 day before said administration, a muscle of the subject, for example the diaphragm, one or more intercostal muscles, quadriceps femoris, and/or the hamstrings, may be injured by injection, preferably a single intramuscular (i.m.) injection, with BaCl₂, notexin, cardiotoxin and/or bupivacaine. The effect of bupivacaine is mild in comparison to the other compounds and is FDA-approved in humans.

As an alternative, or in addition, a muscle of the subject may be injured by freezing, which will cause a local injury, or by a single local irradiation at about 18 Gy. During irradiation, the subject will be protected by a lead shield, and only one, or a part of one muscle, may be irradiated.

As an alternative, or in addition, a muscle of the subject may be injured by exercise training, for example on a threadmill using either a short, intense protocol or a longer, low-intensity protocol. The short, high-intensity exercise protocol may exist of a single 90 minute bout of downhill running (−20 degree angle decline) on a treadmill, for example at a speed of 15 m/minute from 0 to 60 minutes, 18 m/minute from 60 to 75 minutes, and 21 m/minute from 75 to 90 minutes. Mild electrical stimulus may be provided as motivation to complete the protocol via a shock-grid at the end of the treadmill belt. The long, low-intensity protocol may consist of 1 h/day for 5 days/wk for one month at belt speed >10 m min-1. The duration and speed of the resistance exercise protocol may be adapted to adjust for age of the subject, the severity of the symptoms, and/or for optimizing engraftment efficiency. Exercise as a means to induced muscle regeneration is considered mild and not invasive and can be applied in clinical settings.

### EXAMPLES

### Example 1. Gene-Editing of Pluripotent Stem Cells and Splicing of Expression of GAA in Said Cells

Methods

Construction of the GAA Exon 1-3 Minigene

Genomic GAA DNA region of GAA exon 1-3 (chr17:80101704-80105894, GRCh38.p7) from a healthy control was amplified with Phusion® High-Fidelity PCR Kit (Thermo Scientific, Waltham, Mass.) and cloned into pcDNA3.1(−)Myc-His A vector using XbaI and NotI restriction sites, 1.9 kb of intron 1 was removed by using BsmBI and SbfI restriction sites, followed by a Klenow reaction and a ligation step. The IVS1 variant (c.−32−13T>G) was introduced using the QuikChange II Site-Directed Mutagenesis Kit (Agilent Technologies). Final constructs were validated with restriction enzyme analysis and sequencing.

Culture of HEK293T Cells

HEK293T cells were cultured in DMEM High glucose (Gibco, Waltham. Mass.) supplemented with 10% Fetal Bovine Serum (Thermo Scientific, Waltham, Mass.) and 100 U/ml penicillin streptomycin (Gibco, Waltham, Mass.). HEK293T cells were plated with 40.000 cells in 12 wells and 24 hours later transfected with 1 μg plasmid using Fugene 6 (Promega, Fitchburg. Wis.) transfection reagent according to manufacture's manual. RNA was harvested after 72 hours of transfection.

Culture and Differentiation of iPSCs into MPCs

Reprogramming and feeder-based culture of iPSCs was performed according to Warlich et al. (Warlich et al., 2011), iPSCs were differentiated into myogenic progenitor cells (MPCs) and expanded as described in van der Wal et al (van der Wal et al., 2017). Briefly, differentiation of iPSCs into myogenic progenitor cells (MPCs) was started on 10 cm dish with a 40% confluent culture, grown for 5 days in iPSC medium followed by the myogenic differentiation procedure consisting of: 5 days myogenic differentiation medium (DMEM/F12, 1% Insulin-Transferrin-Selenium-Ethanolamine, 1% penicillin/streptomycin/L-glutamine, all Gibco, Waltham, Mass.), supplemented with 3.5 μM CHIR99021 (Axon Medchem, Groningen, The Netherlands), 14 days myogenic differentiation medium supplemented with 20 ng/ml FGF2 (Prepotech, Rocky Hill, N.J.) and 16 days myogenic differentiation medium only. For purification, MPCs were sorted with FACS with anti-C-MET-APC (1:50, R&D systems, Minneapolis Minn.) and anti-HNK-1-FITC (1:100, Aviv Systems Biology, San Diego, Calif.) antibodies. Purified MPCs were expanded in MPCs proliferation medium consisting of DMEM high glucose supplemented with 10% Fetal Bovine Serum, 100 U/ml penicillin/streptomycin and 100 ng/ml FGF2. Differentiation of MPCs into skeletal muscle cells was started when MPCs reached a confluency of 90%, by switching to myogenic differentiation medium. After 4 days skeletal muscle cells were harvested.

Gene Editing of iPSCs

Single guide RNA (sgRNA) sequences were designed using CRISPRscan program (Moreno-Mateos et al., 2015) to select optimal target sites for intron 1 (table 1). Selected sgRNA sequences were inserted in a TOPO vector containing the U6 promoter (addgene: 41824). Prior to gene editing of iPSCs, all sgRNAs were first tested in HEK293T cells. Confluent iPSCs on feeders were pretreated 4 hours before nucleofection with 10 μM Rock inhibitor (Y-27632 dihydrochloride, Ascent Scientific, Asc-129). Single cells were generated from iPSC colonies by incubating with Accutase (Thermo Scientific, Waltham, Mass.), and 2*106 cells were nucleofected with 3.6 μg pCAG-hCAS9-GFP (addgene: 44719) and 1.8 μg of each TOPO-sgRNA using Amaxa Human Stem Cell Nucleofector Kit2 (VPH-5022) with program B-016. After nucleofection, cells were recovered in iPSC-conditioned medium (iPSC medium incubated for 24 hours on feeder cells) supplemented with 20 ng/ml FGF2 and 10 μM rock inhibitor. After 48 hours, single iPSCs were plated in a dilution range to obtain single iPSC colonies.

Genotyping of Single iPSC Colonies

Approximately, 14 days after plating single iPSCs, single colonies were picked and expanded in 2×48 wells containing feeders. After 7 days, 1 well was sacrificed for genotyping. Genomic DNA was extracted using a high salt method. Positive iPSC clones were expanded and tested. PCR and sequencing was used to determine the purity of iPSC clones, and to identify the targeted allele and the position of the cleavage site. The PCR reaction consisted of 12.5 ng genomic DNA with 0.4 μL Faststart Taq Polymerase (Roche, Penzberg, Germany), 0.33 mM dNTPs, 0.33 μM forward and reverse primers in a 15 μL reaction (table 2).

RNA Isolation and cDNA Synthesis

RNA was isolated using the RNeasy minikit with DNAse treatment (Qiagen, Germantown, Md.). Isolated RNA was stored at −80° C. 500 ng of RNA was synthesized into cDNA using the iScript cDNA synthesis kit (Bio-Rad, Hercules, Calif.) according to manufacturer's protocol.

qRT-PCR

Quantitative reverse transcriptase polymerase chain reaction (qRT-PCR) was performed with a CFX96 real-time system (Bio-Rad, Hercules, Calif.). cDNA was diluted 5× or 10× times and 4 μL was used in a qRT-PCR reaction consisting of a total volume of 15 μL with 7.5 μL iTaq Universal SYBR Green Supermix (Bio-Rad, Hercules, Calif.), 10 pmol/μl forward and reverse primers (Table 2). For each plate a standard curve was included with 5 dilutions.

Flanking Exon RT-PCR

Flanking RT-PCR was performed with 2 μL of 10× diluted cDNA with 10 pmol/μl forward and reverse primers (table 2) using the Advantage GC 2 PCR kit (Clontech. Kusatsu. Japan) with a GC-melt concentration of 0.5 M according to the manufacturer's instructions. A 1.5% agarose gel containing 0.5 μg/ml ethidium bromide (Sigma Aldrich, Irvine, UK) was used to analyze the whole RT-PCR reaction.

GAA Enzyme Activity Assay

Skeletal muscle cells were harvested with ice cold lysis buffer (50 mM Tris (pH 7.5), 100 mM NaCl, 50 mM NaF, 1% Triton X-100 and one tablet Protease Inhibitor Cocktail cOmplete, with EDTA, (Roche. Penzberg, Germany)) for 10 minutes on ice. GAA enzyme activity was measured using 4-methylumbelliferyl α-D-glucopyranoside substrate (Sigma Aldrich, Irvine. UK) as described previously (Kroos et al., 2007). Total protein concentrations was determined with the BCA protein assay kit (Pierce, Thermo Scientific, Waltham, Mass.).

RNA Sequencing

RNA samples were sequenced according to van der Wal et al (van der Wal et al 2017b). Analysis was performed with new Tuxedo pipeline (Pertea et al., 2016). Briefly, FASTQ RNA sequencing files were aligned to HG38 (igenome, Illumina, San Diego, Calif.) using HISAT2 and assembled with Stringtie. Fragments Per Kilobase Million (FPKM) were calculated with the ballgown package.

Immunofluorescence

Cultured skeletal muscle cells were fixed with 4% paraformaldehyde (Merck, Kenilworth, N.J.) in PBS for 10 minutes at room temperature (RT) and stored in PBS at 4° C. For immunofluorescence cells were permeabilized for 5 minutes with 0.1% Triton X-100 (AppliChem, Darmstadt, Germany) in PBS and blocked for 30 minutes at RT with blocking solution (PBS, with 0.1% Tween and 3% BSA, all Sigma Aldrich, Irvine, UK). Rabbit-α-Myogenin (Santa Cruz, sc-.576, 1:100) and Mouse-α-MF20 (DSHB, 1:50) were diluted into 0.1% BSA in PBS-T (PBS with 0.1% Tween) and incubated for 1 hour at RT. After incubation cells were washed (1×PBS-T and 1×PBS both for 2 minutes) and incubated with the secondary antibodies (Alexa-Fluor-488-α-rabbit 1:500. Invitrogen. Carlsbad, Calif. and horse anti-mouse biotin, Vector Laboratories, Burlingame, Calif.) in PBS-T for 30 minutes at RT. For incubations with secondary biotinylated antibodies cells were washed and incubated with Streptavidine 594 (1:500. Invitrogen, Carlsbad, Calif.,). The cells were washed and incubated for 15 minutes with Hoechst (1:15000, Thermo Scientific. Waltham, Mass.) and imaged in PBS.

TABLE 1
Single guide RNAs used to remove
2.1 kb of intron 1

| Position | Sequence ′5-′3 |
|---|---|
| Left | CCGTGGCCTGAGAGGGGGCCCC |
| Right | CCCTGCTGGAGCTTTTCTCGC |

TABLE 2
Primers used for RT-qPCR, RT-PCR and PCR

| Name | Sequence ′5-′3 | Assay |
|---|---|---|
| GAA Exon | AAACTGAGGCACGGAGCG | RT-qPCR |
| 1-2 fw | | |
| GAA Exon | GAGTGCAGCGGTTGCGAA | RT-qPCR |
| 1-2 rv | | |
| GAA | GGCACGGAGCGGGACA | RT-qPCR |
| Cryptic | | |
| Exon 2 fw | | |
| GAA | CTGTTAGCTGGATCTTTG | RT-qPCR |
| Cryptic | ATCGTG | |
| Exon 2 rv | | |
| GAA Full | AGGCACGGAGCGGATCA | RT-qPCR |
| Skip Exon | | |
| 2 fw | | |
| GAA Full | TCGGAGAAGTCGACGCTG | RT-qPCR |
| Skip Exon | TA | |
| 2 rv | | |
| GAA_Exon1_ | AGGTTCTCCTCGTCGGCC | RT-PCR |
| GC_FW1 | CGTTGTTCA | |
| GAA_Exon3_ | TCCAAGGGCACCTCGTAG | RT-PCR |
| GC_RV1 | CGCCTGTTA | |
| Fw_delta_ | CAGAAGCGGGTTTGAACG | PCR, |
| intron1_ | TG | determines |
| bp_seq | | cleavage |
| | | site |
| Rv_delta_ | GGAGAAGAAAGCGGGCTC | PCR, |
| intron1_ | AG | determines |
| bp_seq | | cleavage |
| | | site |
| Fw_in 1_ | TGGGAAAGCTGAGGTTGT | PCR, |
| gDNA | CG | initial |
| | | screening |
| Rv_in 1_ | CAGCTCTGAGACATCAAC | PCR, |
| gDNA | CG | initial |
| | | screening |
| Fw_ | CATGGCTGGGTCTGAATC | PCR, |
| primer_1 | CC | specific |
| | | for Δ |
| | | intron 1 |
| | | product |
| Fw_ | TACCTGCCTTGCTGGTGT | PCR, |
| primer_2 | TC | specific |
| | | for WT |
| | | product |
| Rv_ | GGTGAGTCTCCTCCAGGA | PCR |
| primer_3 | CT | |

Results

Shortening of GAA Intron 1 in a Minigene Increases Expression and Corrects Splicing of GAA in an IVS1 Background

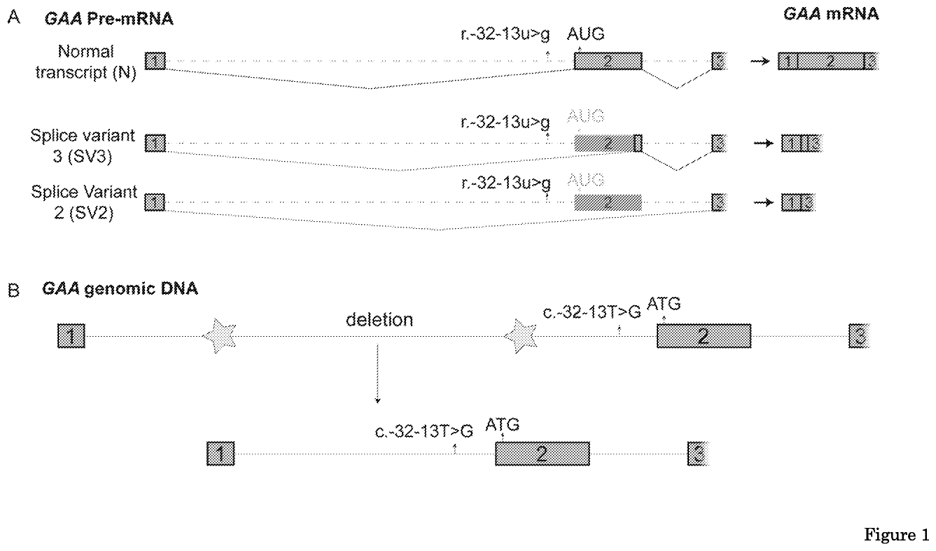

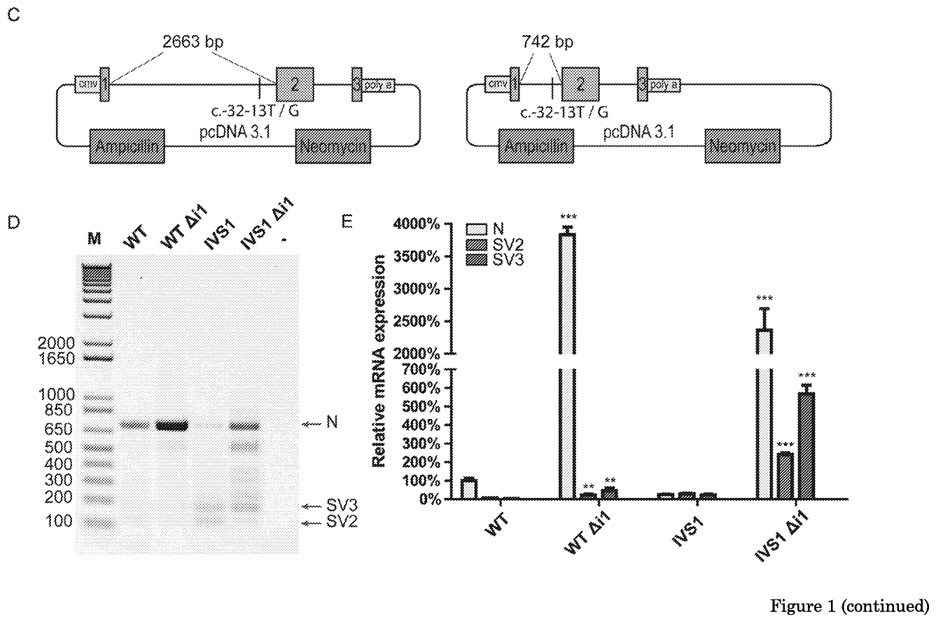

The IVS1 (c.−32−13T>G) variant in Pompe disease is located in the polypyrimidine-tract (pY-tract) of intron 1 of the GAA pre-mRNA, and causes aberrant splicing to several additional splice variants including SV2 and SV3 that do not include an AUG translation start site (FIG. 1A). Roughly ˜10% of the transcripts are correctly spliced, which explains its association with a childhood/adult Pompe disease phenotype (Boerkoel et al., 1995; Dardis et al., 2014: Huie et al., 1994). Previous removal of a large part of intron 1 (2 kb) in a minigene comprising GA4 exon 1 to exon 3 resulted in increased mRNA expression, both in an IVS1 and a wildtpe (WT) background (Raben et al., 1996). This was explained by the removal of a binding site for the transcriptional repressor HES-1 (Yan et al., 2002a; Yan et at, 2002b). However, it was unknown to what extent shortening of intron 1 may affect GAA splicing. To test this, we used a similar minigene that has been described by us previously (van der Wal et al., 2017b), and we deleted 1.9 kb of intron 1 (FIG. 1C). FIG. 1D shows the flanking exon RT-PCR analysis of the transfection of these minigenes in HEK293T cells. Compared to the WT minigene, the IVS1 minigene showed decreased expression of the normal transcript N, while expression of SV2 and SV3 were increased. After the removal of 1.9 kb of intron 1, a higher expression of normal transcript was detected both in WT All and IVS1 All minigenes. To exclude that the elevated expression was the result of changes in transfection efficiency, Neomycin was amplified and showed no significant differences in expression (data not shown). To quantify the expression levels, RT-qPCR was applied with specific primers targeting the N, SV2 and SV3 transcripts (van der Wal et al., 2017) (FIG. 1E). Intron 1 deletion in the WT minigene caused a 38-fold increase in expression of the N variant, while expression of SV2 and SV3 transcripts were increased 4 and 8 fold, respectively. When comparing IVS1 with IVS1 ΔI1 minigenes, intron 1 deletion caused a 94 fold increase in expression for product N, and a 13 and 23 fold increased expression for the SV2 and SV3 variants, respectively. These results suggested that: 1) deletion of a large part of intron 1 increases expression of normal GAA transcript, and 2) in addition to this, deletion of a large part of intron 1 facilitates normal GAA splicing. The latter conclusion can be deduced from the observation that intron 1 deletion induced a larger increase in expression of the SV2 and SV3 in the IVS1 minigene compared to the WT minigene. Taken together, 1.9 kb removal of intron 1 both increased expression and corrected aberrant splicing in GAA minigenes.

Shortening of the Genomic Intron 1 Sequence in Patient-Derived Induced Pluripotent Stem Cells

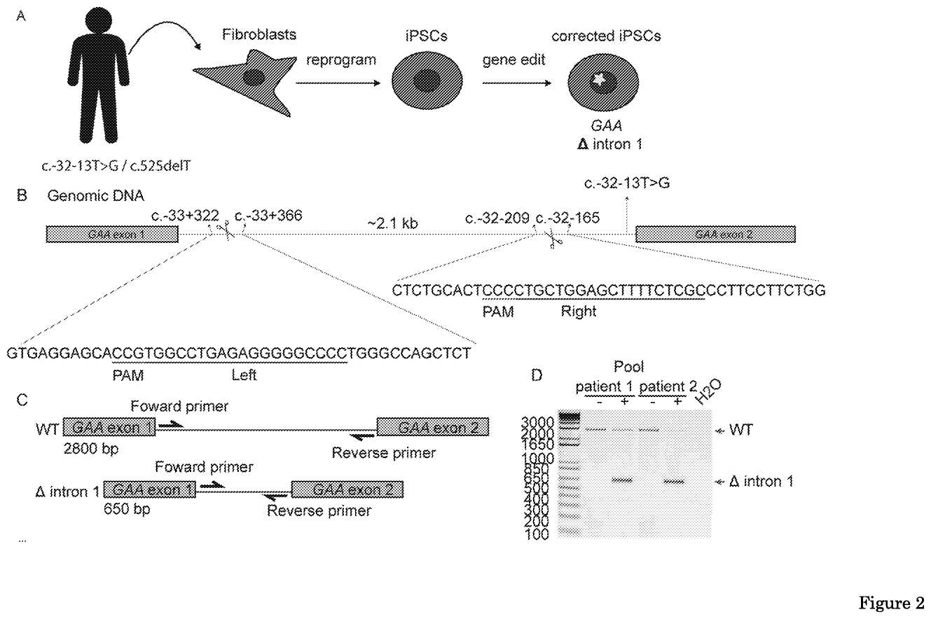

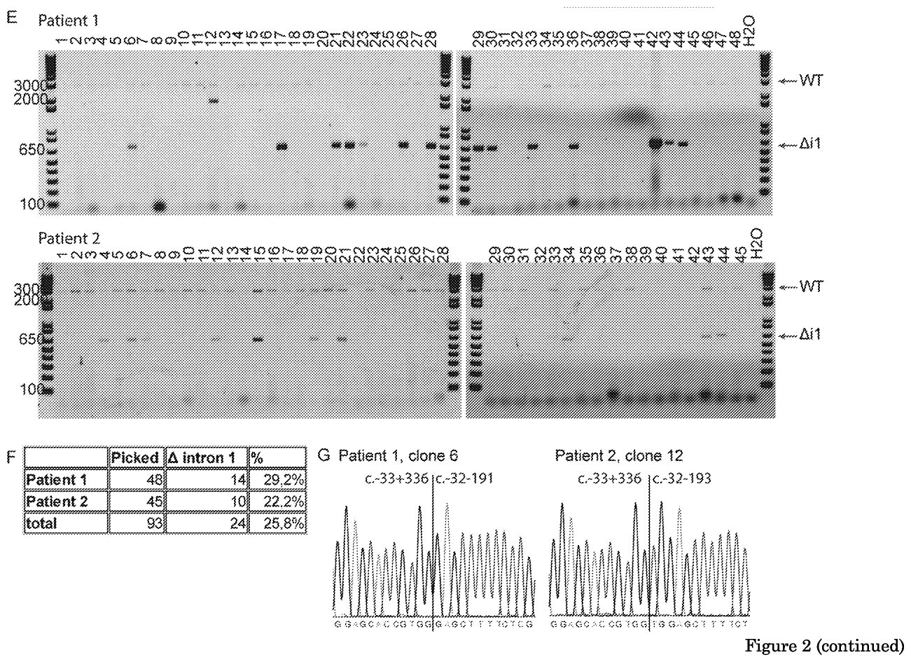

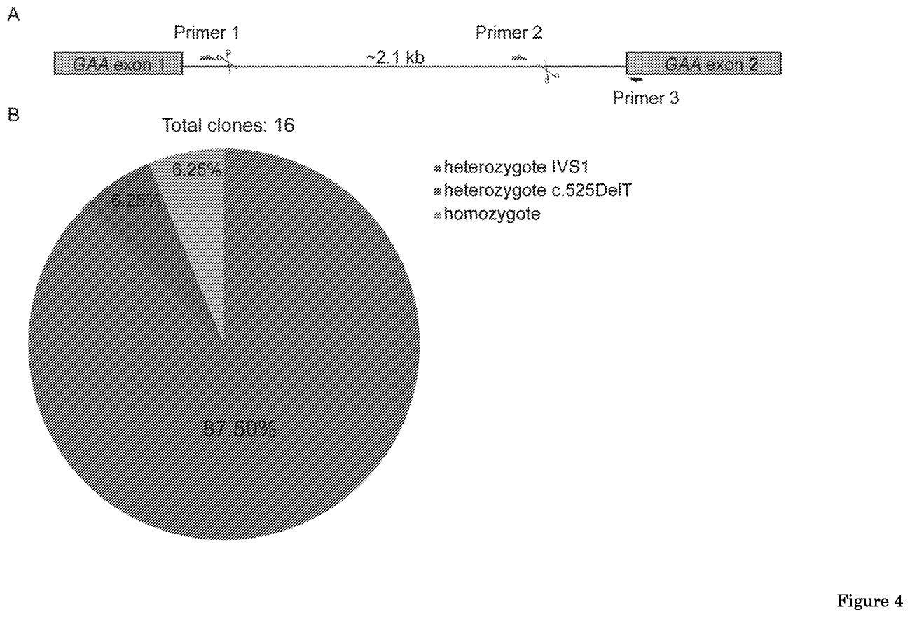

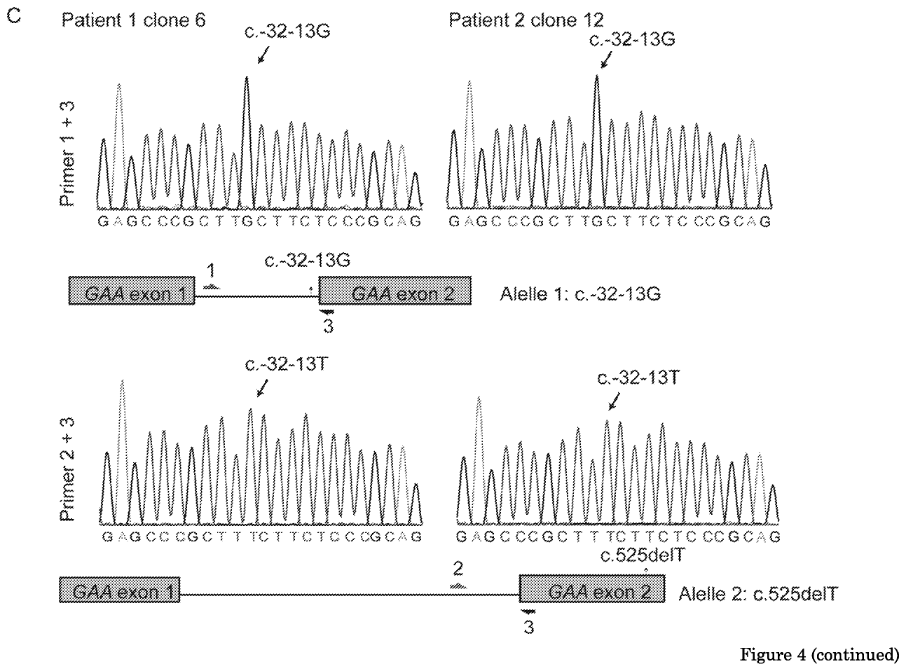

Next, we wished to test the effect of shortening intron 1 in genomic DNA of patient cells. To this end, we reprogrammed 2 fibroblast cell lines from different Pompe patients that carried the IVS1 and c.525delT variant into iPSCs. A lentiviral vector carrying the four reprogramming factors (Oct4, Sox2. KLF4 and C-myc) was used (Warlich et al., 2011). The deleterious c.525delT variant causes degradation of GAA mRNA by a reading frame shift, rendering transcription from the IVS1 allele as the only source of GA4 mRNA, thereby reducing background (Hermans et al., 1994). Two single guide RNA (sgRNA) sequences were selected using the CRISPRscan algorithm (Moreno-Mateos et al., 2015) to delete 2.1 kb of intron 1, and these showed low predicted numbers of off-target hits (<1) (FIG. 2A). PCR screening of iPSC colonies was performed with primers that span the deletion and amplified the full-length (WT) product and the 2.1 kb deleted intron 1 (Δ intron 1) product (FIG. 2C). The Δ intron 1 product was detected in the polyclonal pool after transfection of the CRISPR/Cas9 and sgRNAs vectors in patient 1 and 2 iPSCs. This indicated successful targeting (FIG. 2D). To obtain a pure clone, single colonies were picked and screened. We detected in patient 1 and 2 iPSCs that 14/48 and 10/45, respectively, of picked clones were positive for the Δ intron 1 product, resulting in an average efficacy of 25% (FIGS. 2E and 2F). To determine whether positive clones contained a heterozygote or homozygote deletion, and to detect from which allele intron 1 was partially removed, specific primers amplifying either the WT or Δ intron 1 product were used (FIG. 4A). Analyzing a subset of positive clones (N=16), showed that one clone (6.25%) contained a homozygote deletion, one clone (6.25%) targeted the c.525delT allele, while all the other clones showed a deletion on the IVS1 allele (87.5%). These results indicate a bias towards targeting of the IVS1 allele (FIG. 4B). For each patient iPSC line, a clone was selected that contained the deletion in intron 1 on the IVS1 allele (FIG. 4C). Analysis of the cleavage site of the selected clones showed similar deletions in intron 1, with removal of 2137 bp for patient 1 clone 6 and removal of 2135 bp for patient 2 clone 12 (FIG. 2G). We conclude that shortening of GAA intron 1 in iPSCs using CRISPR/cas9 is a highly efficient procedure.

Shortening of GAA Intron 1 Increases GAA Expression and Corrects Exon 2 Splicing in iPSCs Derived Skeletal Muscle Cells

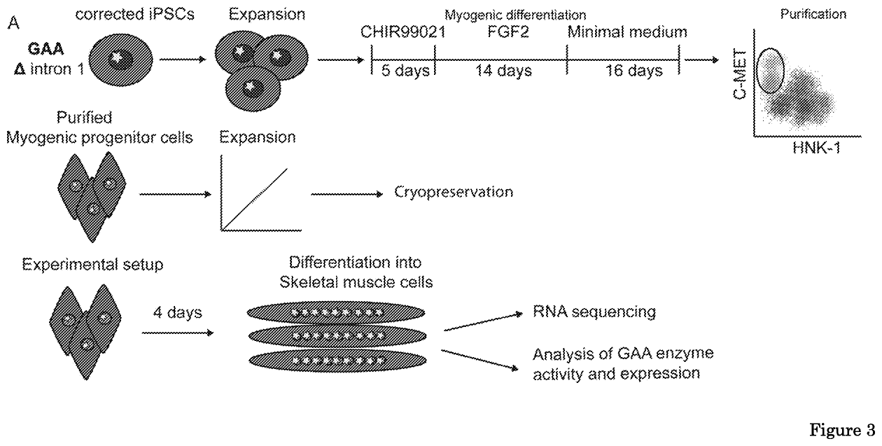

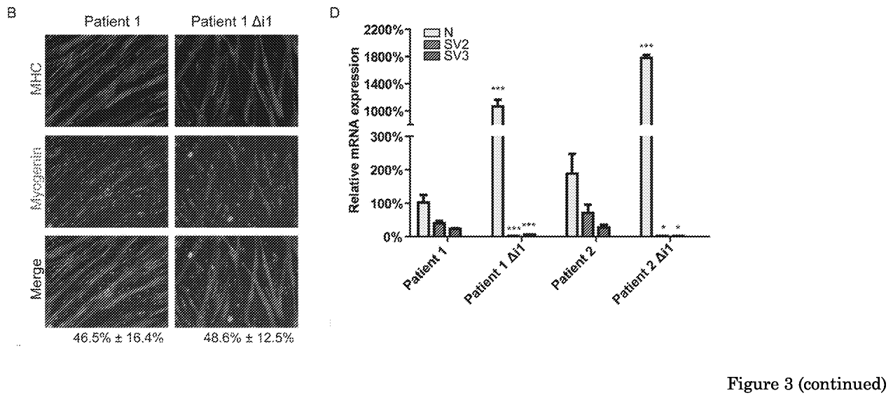

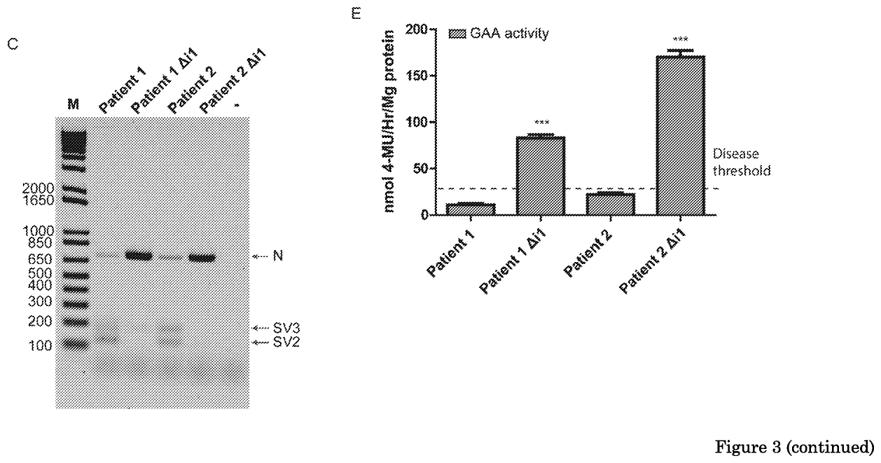

Alternative splicing is a cell-type specific progress and it is not clear whether results obtained with shortening of intron 1 in the context of a minigene can be extended to endogenous GAA expression. We successfully generated two patient iPSC lines that contained a ˜2100 bp deletion in intron 1 of the GAA gene as described above. To generate skeletal muscle cells, iPSC lines were differentiated using transgene free differentiation procedure as described previously (van der Wal et al., 2017). After expansion of iPSCs, myogenic differentiation was initiated and followed by purification of MPCs (FIG. 3A).

Differentiation efficiency of patient 1 and 2 iPSCs into MPCs was not changed after shortening of intron 1 (data not shown). MPCs were subsequently differentiated into multinucleated myotubes. During this differentiation, no difference in morphology and fusion index was seen between WT and Δ intron 1 skeletal muscle cells of patient 1 and 2 (FIG. 3B). GAA splicing analysis using flanking exon RT-PCR of exon 2 showed that expression of the splice variant N was enhanced after shortening of intron 1. SV2 and SV3 were almost undetectable on gel, whereas these products were clearly detectable in WT skeletal muscle cells (FIG. 3C). The splicing product-specific qRT-PCR confirmed the results of FIG. 3C. When comparing WT with Δ intron 1 cells from patient 1 and 2, a 10 fold increased expression for product N, and a 35 fold reduction in expression for product SV2 was observed (FIG. 3D). Reduction of SV3 expression in cells from patient 1 and 2 was 6 and about 70 fold, respectively. These results only partially mimicked the results obtained using the minigene: whereas all splice products showed increased expression upon intron 1 deletion in the minigene, although to different extents, only the N product was increased while SV2 and SV3 splice products were strongly reduced following intron 1 deletion at the genomic level. This indicated that intron 1 deletion showed a relatively large increase in normal splicing. However, the increase in the N product was 9-10 fold. This is higher than can be expected from the correction of a single IVS1 allele, which theoretically can also yield correction up to 4-5 fold (up to 50% of healthy individuals). From this result, we infer that intron 1 deletion also increases GAA expression, besides correcting splicing of exon 2.

We then wondered whether upregulation of expression and correction of splicing of GAA, upon intron 1 deletion in muscle cells also increased GAA enzyme activity. For both patient iPSC derived skeletal muscle cells, an 8-fold increase in GAA enzyme activity was detected, which corresponds to a level from two normal copies of GAA (FIG. 3E). In conclusion, genomic shortening of intron 1 showed an increased GAA expression and correction of aberrant splicing caused by the IVS1 variant in skeletal muscle cells, and resulted in complete restoration of GAA enzyme activity to levels that are 2-fold above those in wild type skeletal muscle cells.

Identification of Changed Gene Expression in Response to GAA Deficiency

We showed that shortening of intron 1 completely restored GAA enzyme activity. No difference in morphology between skeletal muscle cells of WT and Δ intron 1 was observed. In agreement with our previous report, which did not show obvious pathological changes in IVS1 skeletal muscle cells, as determined using biochemical measurements of lysosomal glycogen content, and immunofluorescent analysis of lysosomal size and number (van der Wal et al., 2017). We conclude that lysosomal pathology in skeletal muscle cells from IVS1 patients requires longer periods of time to develop in vitro. We anticipated that changes in expression levels of downstream effectors of GAA deficiency might already be affected at an early stage of childhood/adult Pompe disease. Complete restoration of GAA enzyme activity by shortening of intron 1 allows filtering for genetic variation in isogenic donors. To this end, we generated two genome wide-expression datasets by RNA sequencing: 1) WT and Δ intron 1 skeletal muscle cells from patient 1 (dataset 1), and 2) iPSC derived skeletal muscle cells from 2 healthy controls (control 1 and control 2) and 4 IVS1 patients patient 1, patient 2, patient 3 and patient 4) (dataset 2). For this, genome-wide expression was determined using the new Tuxedo pipeline (Pertea et al., 2016). Pearson correlation showed a strong similarity between WT and Δ intron 1 patient 1 skeletal muscle cells (data not shown). In addition, no clustering was seen between patient 1, 2, 3 and 4 IVS1 skeletal muscle cells suggesting genetic or pathological variation between donors. We detected 364 differently expressed (>2 fold) genes in dataset 1 with a similar number of genes that were up or down regulated. Comparing iPSC derived skeletal muscle cells from 2 healthy controls and 4 IVS1 patients (dataset 2) identified 1226 genes that showed a significant (FDR<0.05) change in gene expression. The overlap between dataset 1 and dataset 2 was determined and resulted in the identification of 41 genes in dataset 3 which also included GAA, indicating correct filtering. In conclusion, by the combination of dataset 1 and dataset 2, we significantly reduced the amount of identified genes from 1226 to 41 genes that were differentially expressed between iPSC derived skeletal muscle cells from healthy controls and IVS1 Pompe patients.

A Molecular Link Between GAA and Skeletal Muscle Cell Function and Development

Next, we wanted to determine whether the filtering strategy was able to enrich the datasets for Pompe-related processes. Ingenuity Pathway Analysis (IPA) was applied on the three datasets. The categories: Skeletal and Muscular System Development and Function and Cardiovascular System Development and Function were detected in all three datasets. After filtering by the combination of datasets 1 and 2, a strong reduction of categories unrelated to Pompe disease were observed (i.e. Cancer). Dataset 2 showed the lowest overlap with dataset 3 with 3/10 categories. These results indicate that using an isogenic pair was beneficial for the filtering of genes that are likely differentially expressed due to genetic variation between donors. Next, we investigated the function of the differentially expressed genes in more detail. IPA revealed that genes that were differently expressed in IVS1 patient skeletal muscle cells in dataset 3 were the strongest associated with the category of Skeletal and Muscular System Development and Function. A high percentage (˜31%) of the initial 41 genes was detected in this category. Most significant functions in this category were muscle contraction, function of muscle and morphology of muscle cells (table 3). Although we were unable to detect changes in morphology or lysosomal pathology in isolated skeletal muscle cells from IVS1 Pompe patients, we observed a change in expression level of genes that are related to skeletal muscle development and functions. This indicates a possible link between GAA deficiency and skeletal muscle dysfunction that might be involved in the pathology of juvenile/adult Pompe disease.

TABLE 3
Top 3 of ‘Diseases or Functions Annotation’
found in the category of ‘skeletal and muscular
system development and function’ with genes from dataset 3.

| Diseases or | | |
|---|---|---|
| Functions Annotation | p-Value | Molecules |
| Muscle contraction | 8.27⁻⁷ | ↑KNCNMA1 |
| | | ↓AGT, C5RP3, GAA, LMOD2, |
| | | MYL3, 5LN |
| Function of muscle | 3.28⁻⁷ | ↑KNCNMA1 |
| | | ↓AGT, C5RP3, GAA, ITGA7, 5LN |
| Morphology of muscle | 9.30⁻⁶ | ↑VCAM1 |
| cells | | ↓AGT, CAPN3, C5RP3, GAA, |
| | | ITGA7, LMOD2 |

### Example 2. Differentiation and Expansion of Pluripotent Stem Cells and Engraftment Thereof in Muscle Tissue

Generation of Induced Pluripotent Stem Cells

Control 1 iPSC line was reprogrammed and characterized as previously described in van der Wal et al (van der Wal et al., 2017). Control 2, control 3, control 4 iPSC lines were a gift from Christian Freund and Christine Mummery and have been characterized previously (Dambrot et al. 201:3), iPSCs were cultured on gamma-irradiated mouse embryonic feeder (MEF) cells in culture medium consisting of DMEM/F12 (Invitrogen. Carlsbad, Calif.), with 20% knock-out serum replacement (Invitrogen, Carlsbad, Calif.), 1% non-essential amino acids (Gibco, Waltham, Mass.), 1% penicillin/streptomycin/L-glutamine (100×, Gibco), 2 mM β-mercaptoethanol (Invitrogen, Carlsbad, Calif.) and 20 ng/ml basic fibroblast growth factor (Peprotech. Rocky Hill, N.J.). The iPSC lines were tested for contamination with mycoplasma using the MycoAlert™ Mycoplasma Detection Kit (Lonza, Walkersville, Md.) regularly. All results in this study were obtained with cultures tested negative. Identity of cell lines used in this study was confirmed by DNA sequencing.

Generation and Expansion of Myogenic Progenitor Cells from iPSCs

A confluent iPSC culture in a 35 mm dish was incubated for 5 minutes at 37° C. with 2 mg/ml collagenase IV in DMEM/F12, cut into pieces with a 23 gauge needle, detached with a cell scraper and split into a 10 cm dish with a confluency of 40%. After 5 days, differentiation into myogenic progenitor cells (MPCs) was started with myogenic differentiation medium (DMEM/F12, 1% Insulin-Transferrin-Selenium-Ethanolamine, 1% penicillin/streptomycin/L-glutamine) supplemented with 3.5 μM CHIR99021 for 5 days and changed to myogenic differentiation medium supplemented with 20 ng/ml FGF2 (Peprotech. London. UK) for 14 days. The last 16 days, cells were cultured in myogenic differentiation medium only. For FACS purification cells were detached with TrypLE and stained on ice with anti-C-MET-APC (1:50, R&D systems, Minneapolis Minn.) and anti-HNK-1-FITC (1:100, Aviv Systems Biology, San Diego. Calif.) antibodies. Prior to FACS, Hoechst (33258, Life Technologies, Carlsbad, Calif.) was added to stain live cells. The c-MET⁺/Hoechst⁺/Hnk-1⁻ fraction was sorted in MPC proliferation medium (DMEM high glucose supplemented with 10% fetal bovine serum, 1% penicillin/streptomycin/L-glutamine and 100 ng/ml FGF2) supplemented with 1× Revitacell supplement (Gibco, Waltham. Mass.). For slightly positive cultures, MPCs were sorted with a maximum sorting time of 20 minutes and pooled into a single well until amount of 20.000 for 96 well, 40.000 for 48 well or 80.000 for 24 well was reached. After 24 hours. MPCs were expanded in MPC proliferation medium or cryopreserved. Further details are described previously (van der Wal et al., 2017).

RNA Isolation and RNA Sequencing

MPCs were expanded for 8 passages and harvested either in proliferating conditions or after 4 days of differentiation as described previously (van der Wal et al., 2017). RNA was extracted using the RNeasy minikit with DNAse step (Qiagen. Germantown, Md.). RNase-free water was used to elute RNA, which was stored at −80° C. Sequencing libraries were prepared using TruSeq Stranded mRNA Library Prep Kit (Illumina, San Diego, Calif., USA) according to the manufacturer's instructions. These libraries were sequenced on a HiSeq2500 sequencer (Illumina, San Diego. Calif., USA) in rapid run mode according to the manufacturer's instructions. Reads were generated of 50 base-pairs in length. RNA sequencing datasets listed in table 4 were downloaded and aligned with the datasets generated in this study using the Tuxedo pipeline (Pertea et al., 2016).

Maturation of Myogenic Progenitors into Muscle Fibers

When MPCs reached 90% conlfuence, cells were switched to MPCs differentiation medium containing DMEM High glucose supplemented with 1% penicillin/streptomycin/L-glutamine, 1× insulin/transferin/ethanolamine/selenium and 1% knockout serum replacement (all Gibco). Medium was not refreshed during differentiation and cells were either harvested at 6, 8 or 12 days.

TABLE 4
RNA sequencing datasets used in this study

| Data source | Accession | Abbreviation |
|---|---|---|
| ENA | ERR975347 | Activated Muscle Stem Cell 2 |
| ENA | ERR975349 | Activated Muscle Stem Cell 1 P38 |
| | | treated 2 |
| ENA | ERR975346 | Activated Muscle Stem Cell 1 |
| ENA | ERR975348 | Activated Muscle Stem Cell 1 P38 |
| | | treated 1 |

MPCs control 1
MPCs control 2

| | | |
|---|---|---|
| ENA | ERR975345 | Quiescent Muscle Stem Cell 2 |
| ENA | ERR975344 | Quiescent Muscle Stem Cell 1 |

MPCs 4 days differentiated control 1
MPCs 4 days differentiated control 2

| | | |
|---|---|---|
| NCBI | GSM2452280 | Neural stem cell 1 |
| NCBI | GSM2452281 | Neural stem cell 2 |
| NCBI | GSM2452282 | Neural stem cell 3 |
| ENCODE | ENCBS476ENC | Dermal Fibroblast 1 |
| ENCODE | ENCBS459ENC | Mesenchymal stem cell 2 |
| ENCODE | ENCSR828TEI | Primary Myotube 1 |
| ENCODE | ENCBS018ENC | Chrondocyte 1 |
| ENCODE | ENCLB014ZZZ | Cardiomyocyte |
| ENCODE | ENCBS460ENC | Mesenchymal stem cell 1 |
| ENCODE | ENCSR000CUI | Muscle stem cell 2 |
| ENCODE | ENCSR000AAG | Smooth muscle cell |
| NCBI | SRX689200 | Primary hepatocytes 2 |
| ENCODE | ENCSR000CUI | Muscle stem cell 1 |
| ENCODE | ENCBS019ENC | Chrondocyte 2 |
| ENCODE | ENCBS475ENC | Dermal Fibroblast 2 |
| ENCODE | ENCBS945YXY | Primary Kidney epithelial cell 2 |
| NCBI | SRX673854 | Primary hepatocytes 1 |
| ENCODE | ENCBS007YZP | Primary Kidney epithelial cell 1 |
| ENCODE | ENCSR828TEI | Primary Myotube 2 |
| ENCODE | ENCSR444WHQ | Primary Myoblast 2 |
| ENCODE | ENCBS293AAA | Embryonic stem cell 1 |
| ENCODE | ENCBS624XJG | Embryonic stem cell 2 |
| ENCODE | ENCSR444WHQ | Primary Myoblast 1 |
| ENCODE | ENCBS485ENC | Hematopoietic stem cell |

TABLE 5
Antibodies used in experiments

| Name | Dilution | Company |
|---|---|---|
| Mouse-α-MF20 | 1:50 | DSHB |
| Rabbit-α-Myogenin | 1:100 | Santa Cruz (sc-576) |
| Rabbit-α-MyoD | 1:100 | Santa Cruz (sc-304) |
| Mouse-α-Pax7 | 1:100 | DSHB |
| Mouse-α-Titin | 1:50 | DSHB |

Immunofluorescent Analysis of In Vitro Differentiation

Differentiated cells were fixed by adding 1 volume of 4% paraformaldehyde (PFA) (Merck, Kenilworth, N.J.) in PBS to the medium. PFA was incubated for 10 minutes at room temperature and afterwards replaced by PBS. Fixed cells were either stored at 4° C. or directly stained. For staining, cells were permeabilized for 5 minutes with 0.1% Triton X-100 (AppliChem, Darmstadt, Germany) in PBS and blocked for 30 minutes at room temperature in blocking solution (PBS-T (0.1% Tween. Sigma Aldrich, Irvine. UK) with 3% BSA (Sigma Aldrich. Irvine, UK)). Primary antibodies (Table 5) were incubated 1 hour at room temperature and diluted into 0.1% BSA in PBS-T. The unbound first antibody was removed by washing three times for 5 minutes with PBS-T. Secondary antibodies (1:500, Alexa-Fluor-594-α-goat, Alexa-Fluor-488-α-mouse, Alexa-Fluor-594-α-rabbit, Alexa-Fluor-488-α-rabbit, Invitrogen, Carlsbad, Calif. or horse anti-mouse biotin, Vector Laboratories, Burlingame, Calif.) were incubated for 30 minutes at room temperature in PBS-T. When a secondary biotinylated antibody was used cells were washed three times for 5 minutes with PBS-T and incubated with Streptavidine 594 (1:500. Invitrogen, Carlsbad. Calif.,). The cells were subsequently washed two times for 5 minutes with PBS and incubated for 15 minutes with Hoechst (1:15000. Thermo Scientific, Waltham, Mass.). Cells were imaged in PBS.

Results

Optimization of CHIR99021 Concentration for Differentiation of iPSCs into Myogenic Progenitor Cells

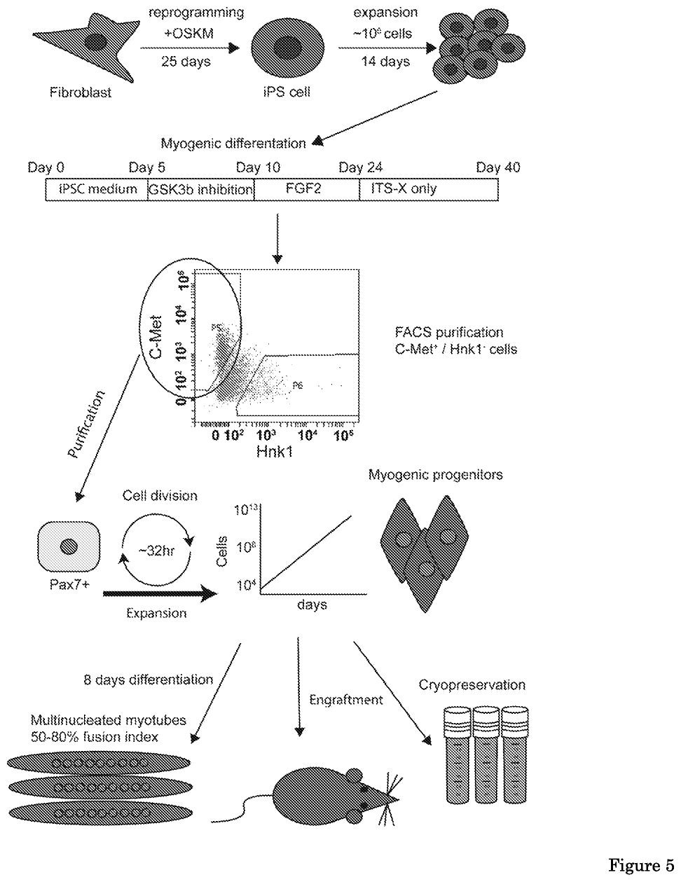

In our previous study, we generated expandable MPCs from four iPSC lines and showed differentiation into multinucleated skeletal muscle cells (van der Wal et al., 2017). The first step of the differentiation procedure from iPSCs into MPCs is the stimulation of the Wnt pathway with CHIR99021. In a previous study on which the current protocol was based. Borchin and colleagues described toxicity at concentrations higher than 3 μM CHIR99021, while Shelton et al. reported that 10 μM CHIR99021 incubated for 2 days resulted in better induction of paraxial mesoderm (Borchin et al., 2013; Shelton et al., 2014). In order to define the optimal concentration of CHIR99021 we generated single cells from iPSCs, and incubated these with 10 μM CHIR99021 for 2 days, followed by the differentiation procedure highlighted in FIG. 5. After 40 days of differentiation, Pax7 positive cells were observed (data not shown). Generating single iPSCs in feeder-based culture systems causes massive apoptosis and addition of Rock inhibitor is required (Watanabe et al., 2007). To keep the differentiation protocol applicable for feeder-dependent and feeder-free cultures we decided to further optimize the concentration of CHIR99021 on plated iPSC colonies with a range from 3 to 5 μM CHIR99021, with incubation for 4, 5, 8 or 10 days. Pax7 staining of control 1 and control 2 iPSCs showed that a concentration of 4 μM of CHIR99021 for 5 days resulted in the highest amount of Pax7-positive areas in the culture (table 6). Elevated concentrations of CHIR99021 (>4 μM) resulted in higher toxicity levels and a more variable response among the different iPSC lines used in this study. To avoid toxicity, we used a concentration of 3.5 μM CHIR99021 in our final differentiation protocol.

TABLE 6
Optimization of CHIR99021 concentration

| Control 1 | | | | Control 2 | | | |
|---|---|---|---|---|---|---|---|
| CHIR99021 | Days | Confluency | Pax7+ | CHIR99021 | Days | Confluency | Pax7+ |
| 3 μM | 4 | 65% | + + | 3 μM | 4 | 95% | + |
| 3 μM | 5 | 50% | + | 3 μM | 5 | 100% | + |
| 3 μM | 8 | 40% | + | 3 μM | 8 | 90% | + |
| 3 μM | 10 | 40% | − | 3 μM | 10 | 95% | + |
| 4 μM | 4 | 85% | + + + | 4 μM | 4 | 95% | + + |
| 4 μM | 5 | 100% | + + + | 4 μM | 5 | 95% | + + |
| 4 μM | 8 | 75% | + | 4 μM | 8 | 95% | + + |
| 4 μM | 10 | 50% | + + | 4 μM | 10 | 85% | + |
| 5 μM | 4 | 60% | + + | 5 μM | 4 | 95% | + |
| 5 μM | 5 | 70% | + + + | 5 μM | 5 | 95% | + + |
| 5 μM | 8 | 0% | − | 5 μM | 8 | 50% | + |
| 5 μM | 10 | 0% | − | 5 μM | 10 | 30% | − |

Evaluation of Robustness of the Myogenic Differentiation Protocol

Previous protocols did not determine the degree of heterogeneity in differentiation and expansion efficiency of iPSC-derived myogenic progenitor cells. We wished to determine the robustness of our optimized differentiation protocol. In addition, we aimed to evaluate whether the protocol could yield expandable MPCs from additional iPSC donor lines. We applied the differentiation procedure highlighted in FIG. 5 in more than 50 individual differentiation experiments using iPSCs from 15 different donors. Eight of these iPSC lines were derived from healthy individuals, while 7 were from Pompe disease patients, ranging from classic infantile to childhood/adult onset (van der Ploeg and Reuser, 2008)). Large Pax7-positive areas were observed in 4 examples of control iPSCs after 40 days of differentiation with similar efficiencies compared to previously used control iPSC lines (van der Wal et al., 2017). Phase contrast microscopy during the differentiation procedure showed small colonies with a confluency between 20-40% at day 1. After 5 days of culture, iPSC-colonies reached a medium size and at this stage exposure to CHIR99021 was started (data not shown). After 5 days of incubation with CHIR99021, we observed increased cell detachment, which was attenuated after 3-4 days in FGF2 containing medium. From day 17 onwards, the cells started to proliferate rapidly, and cultures became completely confluent after 24 days of culture. Cells with a similar morphology as skeletal muscle cells were observed between 30 and 40 days. We observed similar morphological changes in all iPSC control lines during this differentiation procedure.

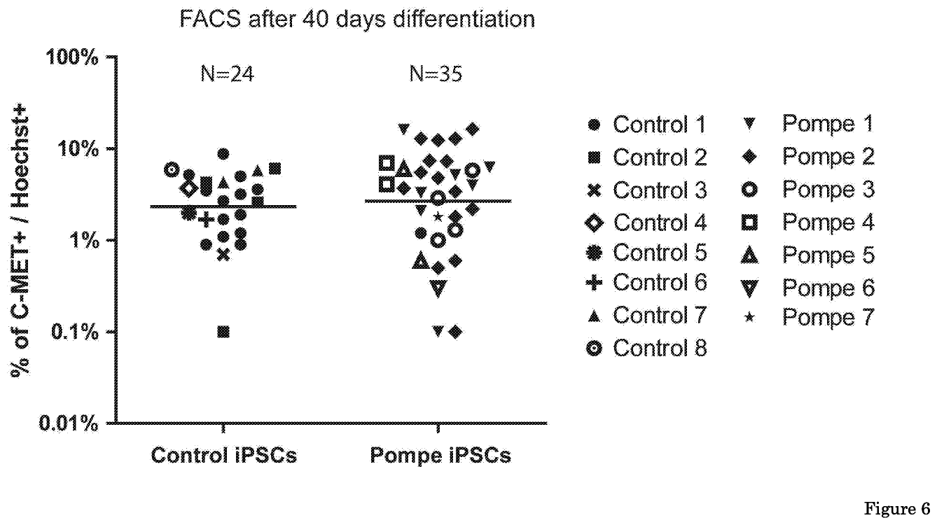

Differentiation of 59 individual cultures of 15 donors yielded on average 4.26%±3.96% of C-MET⁺/Hoechst⁺/HNK-1⁻ positive cells (FIG. 6). No significant differences in amount of C-MET⁺/Hoechst⁺ cells was observed between healthy control and Pompe disease iPSCs. Sorting differentiation cultures with low recovery of C-MET⁺/Hoechst⁺/HNK1-⁻ cells (˜0.2% of cells) resulted in expandable MPCs with similar differentiation capacity compared to cultures with a high recovery (>2%) (data not shown: recoveries of individual experiments are shown in FIG. 6). After 24 hours of plating, sorted MPCs revealed a rather uniform morphology suggesting a homogeneous cell population. These results demonstrated that the differentiation protocol generated C-MET⁺/Hoechst⁺/HNK 1-myogenic cells with high reproducibility.

Expansion and Molecular Characterization of MPCs

We previously determined the proliferation rate of purified MPCs during 31 days of culture (van der Wal et al., 2017). It remained unclear to what extent these MPCs could proliferate and differentiate into multinucleated skeletal muscle cells. To determine expansion capacity, we extended the proliferation phase of control 1 and control 2 MPCs from 31 to 50 days. Similar proliferation rates were observed during a period of up to 40/41 days, reaching respectively 1.5×10¹³ and 1.7×10¹³ cells for control 1 and control 2 MPCs, respectively. After 41 days, the proliferation rate diminished, morphology of cells changed and differentiation capacity was reduced (data not shown). This showed that the MPCs generated with this protocol could be expanded up to a maximum of 108 fold.

We next wished to determine the molecular signature of expanded MPCs and compared this with published signatures of myogenic cells. As determined above, proliferation rates were stable between day 0 and day 40 of expansion and therefore we extracted RNA from control 1 and control 2 MPCs that were expanded for 17 days and either harvested while proliferating or after induction of differentiation for 4 days. RNA sequencing was performed and datasets were compared with publically available RNA sequencing datasets using the Tuxedo pipeline (table 4) (Pertea et al., 2016). MPCs grown in proliferation medium showed a strong correlation with expression profiles of activated muscle stem cells, while MPCs that were differentiated for 4 days clustered in the same tree compared to the proliferating MPCs but correlated to a lesser extent. The non-myogenic cell types did not show any correlation with the proliferating or differentiating MPCs, thus indicating 1) that the differentiation and purification protocol employed here resulted in full entry into the myogenic lineage, and 2) that the MPC culture was of high purity.

Differentiation of Expanded MPCs into Mature Skeletal Muscle Cells

Recently, several studies demonstrated complete maturation with striated titin expression and spontaneous contraction of iPSCs derived skeletal muscle cells (Chal et al., 2015; Choi et al., 2016; Swartz et al., 2016). However, in these studies no purification step was applied and it was suggested that co-culture of neuronal cells might be required for the induction of contractions. To address this, we tested maturation of purified MPCs. Previously, we demonstrated that the differentiation capacity remained stable during the expansion phase of MPCs, using a 4 day differentiation protocol, based on unchanged fusion indexes during expansion (van der Wal et al., 2017). Unfortunately, extended culture of MPC-derived myotubes in conventional differentiation media resulted in increased detachment and cell death, and at day 4 of differentiation, we were unable to detect striation and spontaneous contractions (data not shown). The thin myotube-like morphology at this stage suggested that further maturation of MPC cultures was needed to obtain contracting multinucleated cells. We hypothesized that extended culture would require a supportive differentiation medium to prevent detachment and stimulate survival. Supplementation of the MPCs differentiation medium with 0.5-2% Fetal bovine serum increased the overall survival of the culture but also increased the proliferation rate of mononucleated cells, resulting in overgrowth of the cell culture (data not shown). In contrast, supplementation with 1% KNOCKOUT™ Serum Replacement (Gibco) supported further differentiation of MPCs into skeletal muscle cells from control 1 and control 2 for up to 12 days. Longer differentiation resulted in titin-positive fibers with patterns of striation that spontaneously contracted, showing that functional sarcomeres, the strongest indication of terminal differentiation, were formed.

During the proliferation phase of MPCs, few Pax7-positive cells were detected. However, upon differentiation to multinucleated myotubes we observed a strong induction of the frequency of Pax7-positive cells, indicating that the MPCs have the potential to form differentiated muscle cells as well Pax7-positive muscle stem cells. In conclusion, optimized differentiation conditions revealed enhanced maturation of MPCs into contractile, titin positive skeletal muscle cells, and the formation of Pax7-positive muscle stem cells.

TABLE 7
Comparison of transgene free skeletal muscle differentiation protocols

| | Differentiation | Fold | | | Fusion | |
|---|---|---|---|---|---|---|
| Report | protocol | expansion | Cryopreservation | Duration | index | Engraftment |
| Borchin et al. 2013 | FACS | N.R. | N.R. | 35 days | N.D. | N.D. |
| Xu et al. 2013 | In original plate | N.R. | N.R. | 36 days | N.D. | Yes, unpurified culture |
| Hosoyama et al. 2014 | In original plate | N.D. | N.R. | 42 days | N.D. | N.D. |
| Shelton et al. 2014 | In original plate | N.R. | N.R. | 50 days | N.D. | N.D. |
| Chal et al. 2015 | In original plate | N.R. | N.R. | 50 days | N.D. | N.D. |
| Shelton et al. 2015 | In original plate | 3× | N.R. | 50 days | N.D. | N.D. |
| Choi et al. 2016 | FACS | 10⁵× | Yes | 30 days | 10-15% | Yes unpurified culture |
| Caron et al. 2016 | Pre-plating | 1250× | N.R. | 26 days | N.D. | N.D. |
| Chal et al. 2016 | Pre-plating | N.D. | Yes | 35 days | N.D. | N.D. |
| Swartz et al. 2016 | In original plate | N.D. | Yes | N.D. | N.D. | N.D. |
| van der Wal et al. 2017 | FACS | 10⁸× | Yes | 35 days | 60-80% | N.D. |
| This study | FACS | 10⁸× | Yes | 35 days | N.D. | Yes, purified culture |

N.R: Not Reported
N.D: Not determined

### Example 3. In Vivo Myogenic Potential of MPCs

Experimental Approach

Transplantation of MPCs into NSG Mice

Non-obese diabetic scid gamma (NSG™, Jackson Laboratories) mice of 2-6 months old were used for transplantation studies. Mice (independently of gender) were anaesthetized with isoflurane in oxygen from a vaporizer. Regeneration of skeletal muscle was induced by chemical injury. The endogenous skeletal muscle fibers of the mice were injured via injection with 50 μl of barium chloride (BaCl2) into the tibialis anterior (TA) muscle. Twenty four hours later, dissociated cells were injected into the TA muscle in duplicates (1 female and 1 male). A sham injection was used as a negative control for cell transplantation. Mice were sacrificed 4 weeks after cell transplantation, and their TA muscles harvested. TA muscles were frozen in isopentane cooled in liquid nitrogen and stored at −80° C. until analysis, 10 μm cryosections were obtained at intervals throughout the entire muscle and stored at −80° C. for further immunostaining.

Immunofluorescent Stainings

Engrafted TA muscle cryosections were fixed in ice cold acetone for 5 minutes, followed by a permeabilization step with 0.3% Triton X-100 in PBS for 20 minutes. Samples were incubated with a blocking solution of 20% goat serum (DAKO) and 2% BSA (Sigma-Aldrich) in 0.1% Tween in PBS for 1 hour. Sections were incubated with primary antibodies mouse anti-human lamin A/C (Vector, 1:100) plus mouse anti-human spectrin (Leica, 1:100) co-stained with rabbit anti-laminin (DSHB, 1:100) over night at 4° C. Tissue sections were stained with secondary antibodies goat anti-rabbit (Alexa fluor 488, 1:500, life technologies) and horse anti-mouse biotin (Vector, 1:250) for 1 hour at room temperature, followed by incubation with Streptavidin 594 (Invitrogen, 1:500) for 30 minutes. Sections were incubated with hoechst nuclear staining (Invitrogen, 1:15000) for 10 minutes and mounted with Mowiol medium (Sigma-Aldrich). Images were obtained using confocal microscopy (Zeiss LSM 700).

Results

Engraftment of In Vitro Expanded iPSC-Derived Myogenic Progenitor Cells

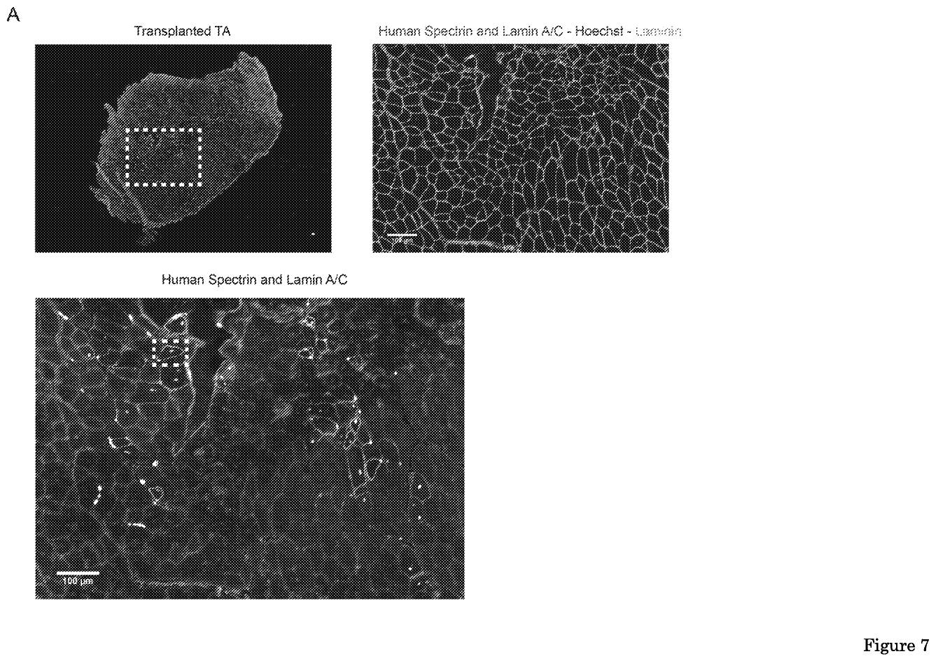

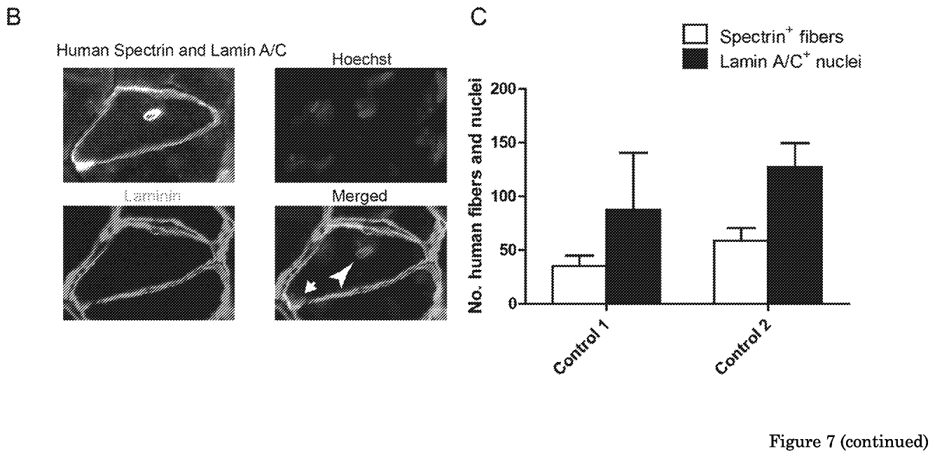

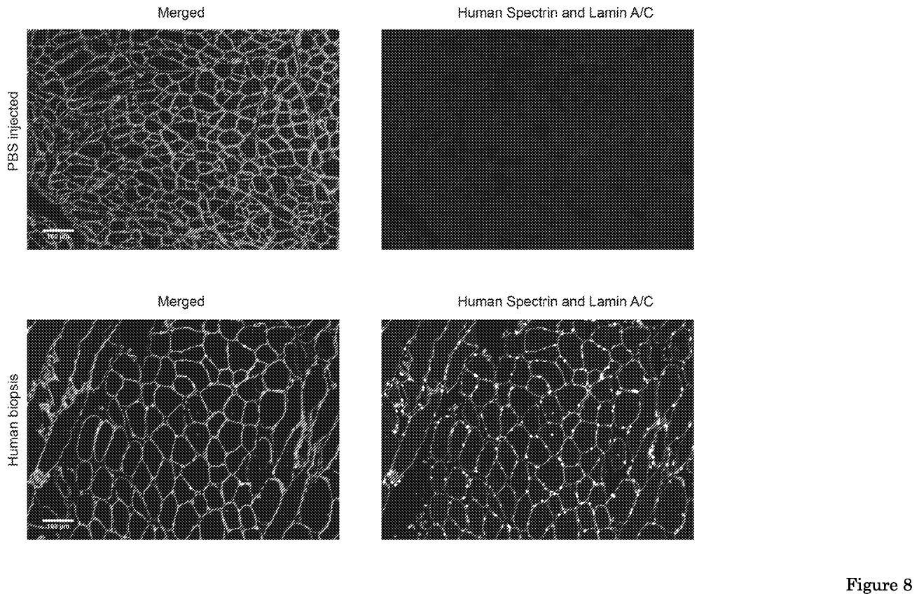

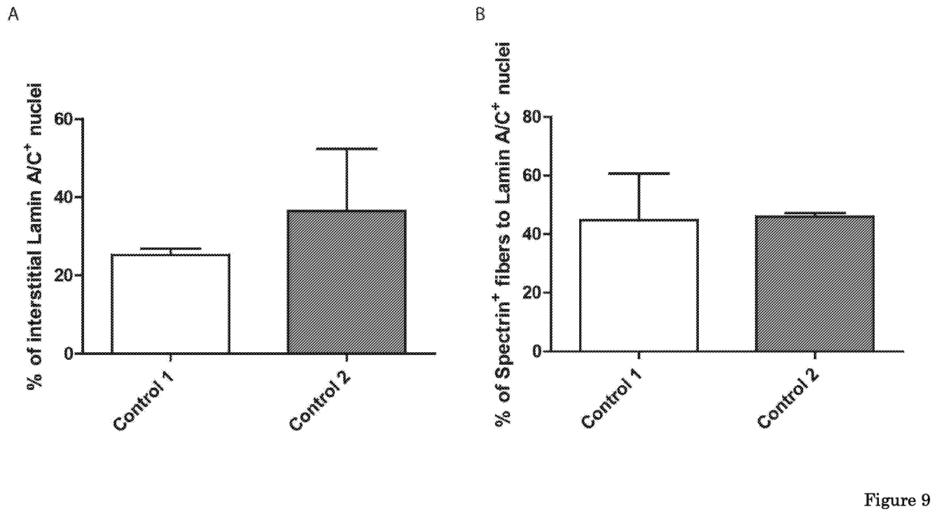

We showed that during proliferation and differentiation MPCs formed Pax7-positive cells, suggesting that the MPCs have the ability to generate muscle stem cells. In addition, transcriptome analysis demonstrated correlation with expression profiles of muscle stem cells. To our knowledge only three studies reported engraftment of transgene-free iPSC-derived MPCs into immune deficient mice using GSK3b-based differentiation protocols (Choi et al., 2016; Kim et al., 2017; Xu et al., 2013) (Table 7). We tested the capacity of purified and expanded MPCs described in this study to engraft and contribute to muscle regeneration in vivo. Human specific antibodies (Lamin A/C and spectrin) were tested on human and mouse muscle sections and showed a positive and specific staining (FIGS. 7 and 8). To this end, we transplanted 5-4×105 iPSC-derived MPCs in tibialis anterior (TA) muscles of immunodeficient recipients that were pre-injured with BaCl2. Cryopreserved MPCs from two different cell lines were expanded over a period of 3 days prior to transplantation. Engraftment efficiency was determined by immunohistochemistry in transplanted muscles 4 weeks post-transplantation. Using human-specific epitopes (lamin A/C and spectrin), we observed that MPCs were able to engraft and participate in the formation of new myofibers (FIGS. 7A and 8). In addition, MPCs engrafted after longer periods of expansion (6 and 11 days) at different cell concentrations (2.5×10\hat{ }5 to 1×10\hat{ }6, control 1 line) (n=6 mice) (data not shown). Engrafted lamin A/C+ nuclei within muscle fibers were located as central myonuclei while others resided beneath the basal lamina (FIG. 7B). Cell engraftment efficiency and muscle contribution was determined by quantifying the number of spectrin+ fibers and lamin A/C+ nuclei (n=2 mice per line transplanted) (FIG. 7C). In addition, we determined the contribution to muscle regeneration as the percentage of spectrin+ fibers compared to total lamin A/C+ nuclei detected in single cross-sections as well as the proportion of lamin A/C+ nuclei present at the muscle interstitium (FIG. 9). These results indicate the engraftment potential and regenerative capacities of expanded MPCs and their possible contribution as multinucleated myofibers or mononuclear cells.

### REFERENCES

Anderson. L. J., Henley. W., Wyatt, K. M., Nikolaou, V., Wahlek, S., Hughes. D. A., Lachmann, R. H., and Logan, S. (2014). Effectiveness of enzyme replacement therapy in adults with late-onset Pompe disease: results from the NCS-LSD cohort study. J Inherit Metab Dis 37, 945-952.
Awaya. T., Kato, T., Mizuno, Y., Chang, H., Niwa. A., Umeda, K., Nakahata, T., and Heike, T. (2012). Selective development of myogenic mesenchymal cells from human embryonic and induced pluripotent stem cells. PLoS One 7, e51638.
Barberi. T., Bradbury, M., Dincer, Z., Panagiotakos, G., Socci, N. I), and Studer, L (2007). Derivation of engraftable skeletal myoblasts from human embryonic stem cells. Nat Med 13, 642-648.
Bigot. A. Jacquemin, V., Debacq, Chainiaux, F., Butler-Browne, G. S., Toussaint, O., Furling, D., and Mouly, V. (2008). Replicative aging down-regulates the myogenic regulatory factors in human myoblasts. Biol Cell 100, 189-199
Boerkoel, C. F., Exelbert, R., Nicastri, C., Nichols, R. C., Miller. F. W., Plotz, P. H., and Raben, N. (1995) Leaky splicing mutation in the acid maltase gene is associated with delayed onset of glycogenosis type II. Am J Hum Gene 56, 887-897.
Borchin. B., Chen. J., and Barberi, T. (2013). Derivation and FACS-mediated purification of PAX3+/PAX7+ skeletal muscle precursors from human pluripotent stem cells. Stem Cell Reports 1, 620-631.
Caron, L., Kher, D., Lee, K. L., McKernan, R., Dumevska, B., Hidalgo, A., Li, J., Yang, H., Main, H., Ferri, G., et al. (2016). A Human Pluripotent Stem Cell Model of Facioscapulohumeral Muscular Dystrophy-Affected Skeletal Muscles. Stem Cells Transl Med 5, 1145-1161.
Chal, J., Al Tanoury, Z., Hestin, M., Gobert., B., Aivio, S., Hick. A., Cherrier, T., Nesmith, A. P., Parker. K. K. and Pourquie, O. (2016). Generation of human muscle fibers and satellite-like cells from human pluripotent stem cells in vitro. Nat Protoc 11, 1833.1850
Chal, J., Oginuma, M., Al Tanoury, Z., Gobert, B., Sumara, O., Hick, A., Bousson, F., Zidouni, Y., Mursch. C., Moncuquet, P., et al (2015). Differentiation of pluripotent stem cells to muscle fiber to model Duchenne muscular dystrophy. Nat Biotechnol 33, 962-969.
Charville, G. W., Cheung, T. H., Yoo, B., Santos, P. J., Lee. G. K., Shrager, J. B., and Rando, T. A. (2015). Ex Vivo Expansion and In Vivo Self-Renewal of Human Muscle Stem Cells. Stem Cell Reports 5, 621-632.
Choi, I. Y., Lim, H., Estrellas, K., Mula, J., Cohen, T. V., Zhang, Y., Donnelly, C. J., Richard, J. P., Kim, Y. J., Kim, H., et al (2016). Concordant but Varied Phenotypes among Duchenne Muscular Dystrophy Patient-Specific Myoblasts Derived using a Human iPSC-Based Model Cell Rep 15, 2301-2312.
Dambrot, C., van de Pas. S., van Zijl, L., Brandl, B., Wang. J. W., Schalij, M. J., Hoeben, R. C., Atsma, D. E., Mikkers, H. M., Mummery, C. L., et al. (2013). Polycistronic lentivirus induced pluripotent stem cells from skin biopsies after long term storage, blood outgrowth endothelial cells and cells from milk teeth. Differentiation 85, 101-109.
Darabi, R., Arpke. R. W., Irion, S., Dimos, J. T., Grskovic, M., Kyba, M., and Perlingeiro, R. C. (2012). Human ES- and iPS-derived myogenic progenitors restore DYSTROPHIN and improve contractility upon transplantation in dystrophic mice. Cell Stem Cell 10, 610-619.
Dardis, A., Zanin, I., Zampieri, S., Stuani, C., Pianta, A., Romananelo, M., Baralle, F. E., Bembi, B., and Buratti, E. (2014). Functional characterization of the common c.−32−13T>G mutation of GAA gene: identification of potential therapeutic agents. Nucleic Acids Res 42, 1291-1302.
de Vries, J. M., van der Beek, N. A., Hop, W. C., Karstens, F. P., Wokke. J. H., de Visser, M., van Engelen, B. G., Kuks, J. B., van der Kooi, A. J., Notermans, N. C., et al. (2012). Effect of enzyme therapy and prognostic factors in 69 adults with Pompe disease; an open-label single-center study. Orphanet J Rare Dis 7, 73.
Goudenege, S., Lebel, C., Huot, N. B., Dufour, C., Fujii, I., Gekas, J., Rousseau, J., and Tremblay, J. P. (2012). Myoblasts derived from normal hESCs and dystrophic hiPSCs efficiently fuse with existing muscle fibers following transplantation. Mol Ther 20, 2153-2167.
Hermans, M. M., De Graaff, E., Kroos, M. A., Mohkamsing, S., Eussen, B. J., Joosse, M., Willemsen, R., Kleijer, W. J., Oostra, B. A. and Reuser, A. J. (1994). The effect of a single base pair deletion (delta T525) and a C1634T missense mutation (pro545leu) on the expression of lysosomal alpha-glucosidase in patients with glycogen storage disease type II, Hum Mol Genet. 3, 2213-2218.
Hockemeyer, D., and Jaenisch, R (2016). Induced Pluripotent Stem Cells Meet Genome Editing. Cell Stem Cell 18, 573-586.
Hosoyama, T., McGivern, J. V. Van Dyke, J. M., Ebert, A. D., and Suzuki, M. (2014). Derivation of myogenic progenitors directly from human pluripotent stem cells using a sphere-based culture. Stem Cells Trans Med 3, 564-574
Huie, M. L., Chen, A. S., Tsujino, S., Shanske, S., DiMauro, S., Engel, A. G, and Hirschhorn, R. (1994). Aberrant splicing in adult onset glycogen storage disease type II (GSDII): molecular identification of an IVS1 (−13T-->G) mutation in a majority of patients and a novel IVS10 (+1GT-->CT) mulation. Hum Mol Genet 3, 2231-2236.
Kaplan, J. C., and Hamroun, D. (2015). The 2016 version of the gene table of monogenic neuromuscular disorders (nuclear genome). Neuromuscul Disord 25, 991-1020.
Krauss, R. S., Joseph, G. A., and Goel, A. J. (2017). Keep Your Friends Close: Cell-Cell Contact and Skeletal Myogenesis, Cold Spring Harb Perspect Biol 9.
Kroos, M. A., Pomponio, R. J., Hagemans, M. L., Keulemans, J. L., Phipps, M., DeRiso, M., Palmer, R. E., Ausems. M. G., Van der Reek, N. A. Van Diggelen, O. P. et al. (2007). Broad spectrum of Pompe disease in patients with the same c.−32−13T->G haplotype. Neurology 68, 110-115.
Lei, Q., Li, C., Zuo, Z., Huang, C., Cheng, H., and Zhou, R. (2016). Evolutionary Insights into RNA trans-Splicing in Vertebrates, Genome Biol Evol 8, 562-577.
Lim, J. A., Li, L., and Raben, N. (2014). Pompe disease: from pathophysiology to therapy and hack again. Front Aging Neurosci 6, 177.
Long, C., Amoasii, L., Mireault, A. A., McAnally, J. R., Li. H., Sanchez-Ortiz, E., Bhattacharyya, S., Shelton, J. M., Bassel-Duby, R., and Olson, E. N. (2014). Postnatal genome editing partially restores dystrophin expression in a mouse model of muscular dystrophy. Science 351, 400-40:3.
Madden, L., Juhas, M., Kraus, W. E., Truskey, G. A., and Bursac, N. (2015). Bioengineered human myobundles mimic clinical responses of skeletal muscle to drugs. Elife 4, e04885.
Maffioletti, S. M., Gerli, M. F., Ragazzi, M., Dastidar, S., Benedetti, S., Loperfido, M., VandenDriessche, T., Chuah, M. K., and Tedesco, F. S. (2015). Efficient derivation and inducible differentiation of expandable skeletal myogenic cells from human ES and patient-specific iPS cells. Nat Protoc 10, 941-958. Kojic, S., Radojkovic, D., and Faulkner, G. (2011). Muscle ankyrin repeat proteins: their role in striated muscle function in health and disease. Crit Rev Clin Lab Sri 48, 269-294.
Maggio. I., and Goncalves, M. A. (2015). Genome editing at the crossroads of delivery, specificity, and fidelity. Trends Biotechnol 33, 280-291.
Moreno-Mateos, M. A. Vejnar, C. E., Beaudoin, J. D., Fernandez, J. P., Mis. E. K., Khokha, M. K., and Giraldez, A. J. (2015). CRISPRscan: designing highly efficient sgRNAs for CRISPR-Cas9 targeting in vivo. Nat Methods 12, 982-988.
Nelson, C. E., Hakim, C. H., Ousterout, D. G., Thakore, P. I., Moreb, E. A., Castellanos Rivera, R. M., Madhavan. S., Pan, X., Ran, F. A., Yan, W. X., et al. (2016). In vivo genome editing improves muscle function in a mouse model of Duchenne muscular dystrophy. Science 351, 403-407.
Palermo, A. T., Palmer, R. E., So, K. S., Oba-Shinjo, S. M., Zhang, M., Richards, B., Madhiwalla, S. T., Finn, P. F., Hasegawa, A, Ciociola, K. M., et al. (2012). Transcriptional response to GAA deficiency (Pompe disease) in infantile-onset patients. Mol Genet Metab 106, 287-300.
Pavlath, G. K. (1996). Isolation, purification, and growth of human skeletal muscle cells. Methods Mol Med 2, 307-317.
Pertea, M., Kim, D., Pertea, G. M., Leek, J. T., and Salzberg, S. L., (2016). Transcript-level expression analysis of RNA-seq experiments with HISAT, StringTie and Ballgown. Nat Protoc 11, 1650-1167.
Raben, N., Nichols, R. C., Martiniuk, F., and Plotz, P. H. (1996) A model of mRNA splicing in adult lysosomal storage disease (glycogenosis type II). Hum Mol Genet 5, 995-1000.
Regnery, C., Kornblum, C., Hanisch, F., Vielhaber, S. Strigl-Pill, N., Grunert, B., Muller-Felber, W., Glocker, F. X., Spranger, M., Deschauer, M., et al. (2012), 36 months observational clinical study of 38 adult Pompe disease patients under alglucosidase alfa enzyme replacement therapy. J Inherit Metab Dis 35, 837-845.
Ruan, G. X., Barry, E., Yu, D., Lukason, M., Cheng, S. H., and Scaria, A (2017). CRISPR/Cas9-Mediated Genome Editing as a Therapeutic Approach for Leber Congenital Amaurosis 10. Mol Ther 25, 331-341.
Ryan, T., Liu, J., Chu, A., Wang, L., Blais, A., and Skerjanc, I. S. (2012) Retinoic acid enhances skeletal myogenesis in human embryonic stem cells by expanding the premyogenic progenitor population. Stem Cell Rev 8, 482.493.
Sato, Y., Kobayashi, H., Higuchi, T., Shimada, Y., Ida, H., and Ohashi, T. (2016a). Metabolomic Profiling of Pompe Disease-Induced Pluripotent Stem Cell-Derived Cardiomyocytes Reveals That Oxidative Stress Is Associated With Cardiac and Skeletal Muscle Pathology, Stem Cells Transl Med.
Sato, Y., Kobayashi, H., Higuchi, T., Shimada, Y., Ida, H., and Ohashi, T. (2016b). TFEB overexpression promotes glycogen clearance of Pompe disease iPSC-derived skeletal muscle. Mol Ther Methods Clin Dev 3, 16054.
Shelton, M., Metz, J., Liu, J., Carpenedo, R. L., Demers, S. P., Stanford, W. L, and Skerjanc, I. S. (2014). Derivation and expansion of PAX7-positive muscle progenitors from human and mouse embryonic stem cells. Stem Cell Reports 3, 516-529.
Shelton, M., Kocharyan, A., Liu. J., Skerjanc I. S., and Stanford. W. L. (2016). Robust generation and expansion of skeletal muscle progenitors and myocytes from human pluripotent stem cells. Methods 101, 73-84.
Shi., Y., Inoue, H., Wu, J. C., and Yamanaka, S. (2017). Induced pluripotent stem cell technology: a decade of progress. Nat Rev Drug Discov 16, 115-130.
Soldner, F., Laganiere, J., Cheng, A. W., Hockemeyer, D., Gao, Q., Alagappan, R., Khurana, V., Golbe, L. I., Myers, R. H., Lindquist, S., et al. (2011). Generation of isogenic pluripotent stem tells differing exclusively at two early onset Parkinson point mutations. Cell 14i, 318-331.
Swartz, E. W., Baek, J., Pribadi, M., Wojta, K. J., Almeida, S., Karydas, A., Gao, F. B., Miller, B. L., and Coppola, G. (2016). A Novel Protocol for Directed Differentiation of C9orf72-Associated Human Induced Pluripotent Stem Cells Into Contractile Skeletal Myotubes. Stem Cells Transl Med 5, 1461-1472.
Tabebordbar, M., Zhu. K., Cheng, J. K., Chew, W. L., Widrick, J. J., Yan, W. X., Maesner, C., Wu, E. Y., Xiao, R., Ran, F. A., et al. (2010). In vivo gene editing in dystrophic mouse muscle and muscle stem cells. Science 351, 407-411.
Takahashi, K., Tanabe, K., Ohnuki, M., Narita, M., Ichisaka, T., Tomoda, K., and Yamanaka, S. (2007). Induction of pluripotent stem cells from adult human fibroblasts by defined factors. Cell 131, 861-872.
Tan, J. Y., Sriram, G., Rufaihah, A. J., Neoh, K. G., and Cao, T. (2013). Efficient derivation of lateral plate and paraxial mesoderm subtypes from human embryonic stem cells through GSKi-mediated differentiation. Stem Cells Dev 22, 1893-1906.
van der Ploeg. A. T., and Reuser, A. J. (2008) Pompe's disease. Lancet 372, 1342-1353
Vafiadaki, E., Arvanitis, D. A., and Sanoudou, D. (2015). Muscle LIM Protein: Master regulator of cardiac and skeletal muscle functions. Gene 560, 1-7.
van der Beek, N. A., Soliman, O. I., van Capelle, C. I., Geleijnse, M. L., Vletter, W. B., Kroos, M. A., Reuser, A. J., Frohn-Mulder, I. M., van Doorn, P. A., and van der Ploeg, A. T. (2008). Cardiac evaluation in children and adults with Pompe disease sharing the common c.−32−13T>G genotype rarely reveals abnormalities. J Neurol Sci 275, 46-50.
van der Beek. N. A., de Vries, J. M., Hagemans, M. L., Hop, W. C., Kroos, M. A., Wokke, J. H., de Visser, M., van Engelen, B. G., Kuks, J. B., van tier Kooi, A. J., et al. (2012). Clinical features and predictors for disease natural progression in adults with Pompe disease: a nationwide prospective observational study. Orphanet J Rare Din 7, 88
van der Ploeg, A. T., and Reuser, A. J. (2008) Pompe's disease. Lancet 372, 1342.1353
van der Wal, E., Bergsma, A. J., van Gtesel, T. J. M., in 't Groen, S. L. M., Zaehres, H., Araúzo-Bravo, M. J., Schöler, H. R., van der Ploeg, A. T., and Pijnappel, W. W. M. P. (2017). GAA Deficiency in Pompe Disease Is Alleviated by Exon Inclusion in iPSC-Derived Skeletal Muscle Cells. Molecular Therapy—Nucleic Acids 7, 101-115.
Warlich, E., Kuehle, J., Cantz, T., Brugman, M. H., Maetzig, T. Galla, M., Filipezyk, A. A., Halle, S., Klump, H., Scholer, H. R., et al. (2011). Lentiviral vector design and imaging approaches to visualize the early stages of cellular reprogramming. Mol Ther 19, 782.789.
Watanabe, K., Ueno, M., Kamiya, D., Nishiyama, A., Matsumura, M., Wataya, T., Takahashi, J. B., Nishikawa, S., Nishikawa, S., Muguruma, K., et al. (2007). A ROCK inhibitor permits survival of dissociated human embryonic stem cells. Nat Biotechnol 25, 681-686.
Xu. L., Park, K. H., Zhao, L., Xu, J., El Refaey, M., Gao, Y., Zhu, H., Ma, J., and Han, R. (2016). CRISPR-mediated Genome Editing Restores Dystrophin Expression and Function in mdx Mice. Mol Ther 24, 564-509.
Xu. C., Tabebordbar, M., Iovino, S., Ciarlo, C., Liu, J., Castiglioni, A., Price, E., Liu, M., Barton, E. R., Kahn, C. R., et al. (2013). A zebrafish embryo culture system defines factors that promote vertebrate myogenesis across species. Cell 155, 909.921.
Yan, B., Raben, N., and Plotz, P. (2002a). The human acid alpha-glucosidase gene is a novel target of the Notch-1/Hes-1 signaling pathway. J. Biol Chem 277, 29760-29764.
Yan, B., Raben, N., and Plotz, P. H. (2002b). Hes-1, a known transcriptional repressor, acts as a transcriptional activator for the human acid alpha-glucosidase gene in human fibroblast cells. Biochem Biophys Res Commun 291, 582-587.
Zaretaky, J. Z., Candotti, F., Boerkoel, C., Adams, E. M., Yewdell, J. W., Blaese, R. M., and Plotz, P. H. (1997). Retroviral transfer of acid alpha-glucosidase cDNA to enzyme-deficient myoblasts results in phenotypic spread of the genotypic correction by both secretion and fusion. Hum Gene Ther 8, 1555-1563

1. Method for gene therapy in a subject suffering from Pompe disease, comprising gene-editing of a glucosidase, acid, alpha gene (GAA) in said subject, which gene-editing comprises removal of a large part of intron 1 of the GAA gene.

2. The method according to claim 1, wherein said subject is suffering from Pompe disease with the GAA IVS1 mutation c.−32−13T>G.

3. The method according to claim 1, wherein said subject is suffering from juvenile or adult forms of Pompe disease, not including the GAA IVS1 mutation c.−32−13T>G.

4. Method for gene therapy in a subject suffering from Pompe disease, comprising the steps of:
providing isolated myogenic progenitor cells, or pluripotent stem cells (PSC), that are obtained from the subject;
removing a large part of intron 1 of the GAA gene by gene editing to provide modified myogenic progenitor cells or PSC;
in case of PSC, differentiate and expand these modified cells to obtain modified myogenic progenitor cells; and
administering said modified myogenic progenitor cells to said subject.

5. Method for treatment of Pompe disease comprising administering to a subject suffering from Pompe disease a cell culture of genetically changed, differentiated myogenic progenitor cells, wherein said cells are derived from said subject suffering from Pompe disease, wherein said cells have been genetically changed by gene-editing, thereby removing a large part of intron 1 of the GAA gene.

6. Method according to claim 1, wherein the gene-editing has been achieved by integrating a viral vector, by action of a site-specific nuclease such as TALEN or ZFN, or by use of a CRISPR based nuclease, such as a Cas9 or Cpf1 enzyme.

7. Method according to claim 4, wherein the Pompe disease is characterized by incorrect splicing of exon 2, in particular wherein the Pompe disease is characterized by the GAA IVS1 mutation.

8. The method according to claim 4, wherein said subject is suffering from juvenile or adult forms of Pompe disease, not including the GAA IVS1 mutation c.−32−13T>G.

9. Method according to claim 1, wherein a large part of the intron 1 of the GAA gene comprises more than 50 of said intron.

10. Method according to claim 4, wherein the differentiation and expansion of the PSC is performed according to the method described in co-pending WO 2017/196175 comprising the steps of:
culturing said PSC in a synthetic culture medium supporting differentiation of said PSC towards a myogenic cell lineage for
(i) a first period of 3-8 days in the presence of between 2-5 microM of CHIR99021,
(ii) a second period of 5-20 days in the presence of 10-30 ng/ml of FGF2; and, optionally,
(iii) a third period of 10-20 days in the presence of insulin-transferrin-selenium-ethanolamine (ITS-X), to thereby provide a cell culture of pre-differentiated PSCs comprising myogenic progenitors cells;
isolating from said cell culture at least one C-Met+ and Hnk1− myogenic progenitor starting cell to thereby provide a purified myogenic cell lineage;
expanding said at least one isolated C-Met+ and Hnk1− myogenic progenitor starting cell in a synthetic culture medium comprising fetal bovine serum (FBS) and 90-110 ng/ml of FGF2 for at least 1 passage to thereby provide a cell culture comprising a population of expanded C-Met+ and Hnk1− myogenic progenitor cells, wherein at least 50% of said population of expanded C-Met+ and Hnk1− myogenic progenitor cells are myogenic marker MyoD positive and myogenic marker Pax7 negative.

11. Method according to claim 1 wherein the administration of the gene-edited myogenic progenitor cells is performed by injection of said cells into a muscle of the subject.

12. Method according to claim 10, wherein the muscle of the subject is injured prior to the administration of the gene-edited myogenic progenitor cells.

13. Vector for use in a method for gene-editing a eukaryotic cell, wherein said vector comprises a guide RNA sequence and wherein said gene-editing comprises removal of a large part of intron 1 of the GAA gene.

14. Vector according to claim 13, wherein said vector encodes a guide RNA targeted to intron 1 of the GAA gene.

15. Myotube prepared from myogenic progenitor cells, wherein said myogenic progenitor cells are characterized by a deletion in the GAA gene resulting from being genetically changed by gene-editing, thereby removing a large part of intron 1 of the GAA gene.

16. Method according to claim 1 wherein said gene-editing is specifically targeted at pluripotent stem cells, myogenic progenitor cells, or myogenic cells.

17. Method according to claim 10 wherein said expanding said at least one C-Met+ and Hnk1− myogenic progenitor starting cell is performed in a culture medium comprising a ROCK inhibitor during at least the culturing period prior to the first passage.

18. Method according to claim 5, wherein the Pompe disease is characterized by incorrect splicing of exon 2, in particular wherein the Pompe disease is characterized by the GAA IVS1 mutation.

19. The method according to claim 6, wherein said subject is suffering from juvenile or adult forms of Pompe disease, not including the GAA IVS1 mutation c.−32−13T>G.

20. Vector according to claim 14, wherein said vector comprises the sequences CCGTGGCCTGAGAGGGGGCCCC and/or CCCTGCTGGAGCTTTTCTCGC.

21. Myotube according to claim 15, wherein said deletion does not cover the basepair sequence c.−32−13T>G responsible for the IVS1 mutation.

22. Myotube according to claim 21 wherein said deletion starts at least 60 nucleotides upstream of the 3′ splice site of exon 2 and at least 10 nucleotides downstream of the 5′ splice site of exon 1.


SEQUENCE LISTING (19 sequences)

SEQ ID NO: 1 - DNA, 22 bp, Artificial Sequence
  gRNA taregt sequence
ccgtggcctgagagggggcccc

SEQ ID NO: 2 - DNA, 21 bp, Artificial Sequence
  gRNA taregt sequence
ccctgctggagcttttctcgc

SEQ ID NO: 3 - DNA, 19 bp, Artificial Sequence
  gRNA target sequence
tggcctgagagggggcccc

SEQ ID NO: 4 - DNA, 18 bp, Artificial Sequence
  gRNA target sequence
tgctggagcttttctcgc

SEQ ID NO: 5 - DNA, 18 bp, Artificial Sequence
  primer
aaactgaggcacggagcg

SEQ ID NO: 6 - DNA, 18 bp, Artificial Sequence
  primer
gagtgcagcggttgccaa

SEQ ID NO: 7 - DNA, 16 bp, Artificial Sequence
  primer
ggcacggagcgggaca

SEQ ID NO: 8 - DNA, 24 bp, Artificial Sequence
  primer
ctgttagctggatctttgatcgtg

SEQ ID NO: 9 - DNA, 17 bp, Artificial Sequence
  primer
aggcacggagcggatca

SEQ ID NO: 10 - DNA, 20 bp, Artificial Sequence
  primer
tcggagaactccacgctgta

SEQ ID NO: 11 - DNA, 27 bp, Artificial Sequence
  primer
aggttctcctcgtccgcccgttgttca

SEQ ID NO: 12 - DNA, 27 bp, Artificial Sequence
  primer
tccaagggcacctcgtagcgcctgtta

SEQ ID NO: 13 - DNA, 20 bp, Artificial Sequence
  primer
cagaagcgggtttgaacgtg

SEQ ID NO: 14 - DNA, 20 bp, Artificial Sequence
  primer
ggagaagaaagcgggctcag

SEQ ID NO: 15 - DNA, 20 bp, Artificial Sequence
  primer
tgggaaagctgaggttgtcg

SEQ ID NO: 16 - DNA, 20 bp, Artificial Sequence
  primer
cagctctgagacatcaaccg

SEQ ID NO: 17 - DNA, 20 bp, Artificial Sequence
  primer
catggctgggtctgaatccc

SEQ ID NO: 18 - DNA, 20 bp, Artificial Sequence
  primer
tacctgccttgctggtcttc

SEQ ID NO: 19 - DNA, 20 bp, Artificial Sequence
  primer
ggtgagtctcctccaggact